SEQUENCE LISTING (8 sequences)

SEQ ID NO: 1 - protein, 520 aa, Homo sapiens
MAALPGTVPRMMRPAPGQNYPRTGFPLEVSTPLGQGRVNQLGGVFINGRPLPNHIRHKIVEMAHHGIRPCVISRQLRVSHGCVSKILCRYQETGSIRPGAIGGSKPRQVATPDVEKKIEEYKRENPGMFSWEIRDRLLKDGHCDRSTVPSGLVSSISRVLRIKFGKKEEEDEADKKEDDGEKKAKHSIDGILGDKGNRLDEGSDVESEPDLPLKRKQRRSRTTFTAEQLEELEKAFERTHYPDIYTREELAQRTKLTEARVQVWFSNRRARWRKQAGANQLAAFNHLLPGGFPPTGMPTLPPYQLPDSTYPTTTISQDGGSTVHRPQPLPPSTMHQGGLAAAAAAADTSSAYGARHSFSSYSDSFMNPAAPSNHMNPVSNGLSPQVMSILGNPSAVPPQPQADFSISPLHGGLDSATSISASCSQRADSIKPGDSLPTSQAYCPPTYSTTGYSVDPVAGYQYGQYGQSECLVPWASPVPIPSPTPRASCLFMESYKVVSGWGMSISQMEKLKSSQMEQFT

SEQ ID NO: 2 - DNA, 2271 bp, Homo sapiens
gaaagctggtgtggagggagaagcgagtgtggtccggagaaagaaggcgtggagaagagggagggagcgagagcgagagaataaatatataaataaatacgagaacgaaatccactccgcagtctccgggctcggaaactttggccccgagcgccagagcgccagagcgcgagagcgcggcgctcgccactctgaggctggcggcctcgattccggccgcgttcccccggcccccctccgccgcggggcctggtctccgggttctgccaggcgcatcagcccgcacaacttctggccgaggccagccggcagaggcggacttggggttggagtgtttgtttgtttgaacttcctcgtcgtcgccaccttccctccccccaacctccaccccacctcacccccctccccagcttctggacgcgtttgactgcagccaggggtggggggtgggggtagggagtgtgtgtggaggggagggagaagaggttaaaaaaaagaagacgaagaagacggaaagaaagagatcgcagcaggggtgaagggagcggacgggaagcgatttttgccgactttggattcgtccccggcgtgcgcaagaatggcggcccttcccggcacggtaccgagaatgatgcggccggctccggggcagaactacccccgcacgggattccctttggaagtgtccaccccgcttggccaaggccgggtcaatcagctgggaggggtcttcatcaatgggcgacccctgcctaaccacatccgccacaagatagtggagatggcccaccatggcatccggccctgtgtcatctcccgacagctgcgtgtctcccacggctgcgtctccaagattctttgccgctaccaggagaccgggtccatccggcctggggccatcggcggcagcaagcccagacaggtggcgactccggatgtagagaaaaagattgaggagtacaagagggaaaacccaggcatgttcagctgggagatccgggacaggctgctgaaggatgggcactgtgaccgaagcactgtgccctcaggtttagtgagttcgattagccgcgtgctcagaatcaagttcgggaagaaagaggaggaggatgaagcggacaagaaggaggacgacggcgaaaagaaggccaaacacagcatcgacggcatcctgggcgacaaagggaaccggctggacgagggctcggatgtggagtcggaacctgacctcccactgaagcgcaagcagcgacgcagtcggaccacattcacggccgagcagctggaggagctggagaaggcctttgagaggacccactacccagacatatacacccgcgaggagctggcgcagaggaccaagctgacagaggcgcgtgtgcaggtctggttcagtaaccgccgcgcccgttggcgtaagcaggcaggagccaaccagctggcggcgttcaaccaccttctgccaggaggcttcccacccaccggcatgcccacgctgcccccctaccagctgccggactccacctaccccaccaccaccatctcccaagatgggggcagcactgtgcaccggcctcagcccctgccaccgtccaccatgcaccagggcgggctggctgcagcggctgcagccgccgacaccagctctgcctacggagcccgccacagcttctccagctactctgacagcttcatgaatccggcggcgccctccaaccacatgaacccggtcagcaacggcctgtctcctcaggtgatgagcatcttgggcaaccccagtgcggtgcccccgcagccacaggctgacttctccatctccccgctgcatggcggcctggactcggccacctccatctcagccagctgcagccagcgggccgactccatcaagccaggagacagcctgcccacctcccaggcctactgcccacccacctacagcaccaccggctacagcgtggaccccgtggccggctatcagtacggccagtacggccagagtgagtgcctggtgccctgggcgtcccccgtccccattccttctcccacccccagggcctcctgcttgtttatggagagctacaaggtggtgtcagggtggggaatgtccatttcacagatggaaaaattgaagtccagccagatggaacagttcacctaaaatgacactgagttgggcaaaacccaggacatctcctggctaagcctctgcttccgtactatggctccaacagaaataaaatacacaacacaaatatcaaaaaaaaaaaa

SEQ ID NO: 3 - protein, 503 aa, Mus musculus
MAALPGAVPRMMRPGPGQNYPRTGFPLEVSTPLGQGRVNQLGGVFINGRPLPNHIRHKIVEMAHHGIRPCVISRQLRVSHGCVSKILCRYQETGSIRPGAIGGSKPRQVATPDVEKKIEEYKRENPGMFSWEIRDRLLKDGHCDRSTVPSVSSISRVLRIKFGKKEDDEEGDKKEEDGEKKAKHSIDGILGDKGNRLDEGSDVESEPDLPLKRKQRRSRTTFTAEQLEELEKAFERTHYPDIYTREELAQRTKLTEARVQVWFSNRRARWRKQAGANQLAAFNHLLPGGFPPTGMPTLPPYQLPDSTYPTTTISQDGGSTVHRPQPLPPSTMHQGGLAAAAAAADTSSAYGARHSFSSYSDSFMNPGAPSNHMNPVSNGLSPQVMSILSNPSAVPPQPQADFSISPLHGGLDSASSISASCSQRADSIKPGDSLPTSQSYCPPTYSTTGYSVDPVAGYQYSQYGQTAVDYLAKNVSLSTQRRMKLGEHSAVLGLLPVETGQAY

SEQ ID NO: 4 - DNA, 4275 bp, Mus musculus
agagacgccaagaggtttatccagccgactctggattcgtctccagcgtgcgcaggaatggcggcgctgcccggcgcggtccccaggatgatgagacccggcccggggcagaactacccgcgcaccggcttccccctggaagtgtccacccctcttggccaaggccgggtcaatcagcttggtggggtcttcatcaacggtcgacccctgccgaaccacatccgtcacaagatagtggaaatggcccaccatggcatccggccctgcgtcatctcccgtcagctccgtgtttcccatggttgtgtctccaagattctgtgccgatatcaggagactgggtccatccggcccggggctatcggaggcagcaagcccagacaggtggcgactccggatgtggagaaaaagattgaggagtataagagagagaaccccgggatgttcagctgggaaatccgggaccggctgctgaaggacggtcactgcgaccgaagcacggtgccctcagtgagttcgattagccgagtgctcagaatcaagttcgggaagaaagaggacgacgaggaaggagacaagaaagaagaagatggcgagaagaaagccaaacacagcatcgatggcatcctgggcgacaaagggaaccgtctggatgagggctcagatgtggaatcagaacccgacctccccctgaagcgcaagcagcgccgcagtcggaccacgttcacagccgagcagctggaggagctggagaaggccttcgagaggacccactacccggacatctacacccgggaggagctggcacagaggaccaagctcacggaggcacgcgtccaggtctggttcagtaaccggcgtgcccgctggcgtaagcaggcaggagctaaccagctggccgccttcaaccaccttctgccgggaggtttcccacccaccggcatgcccacgctgccaccctaccagctgccggactctacctaccccaccaccaccatctcccaagatggaggcagcacagtacacaggccccagccccttccgccatcaaccatgcatcagggtgggctggctgcggccgctgcagcagcggacaccagctctgcctacggagcccgccacagcttctccagctactctgacagcttcatgaaccctggggctccctccaaccacatgaaccctgtcagcaatggcctgtctcctcaggtcatgagcatccttagcaacccgagtgccgtgcctccacagccccaggccgacttctccatctccccgctgcatggaggcctggactcggcttcctccatctcagccagctgcagccaacgggccgactccatcaagccaggagacagcttgcccacgtcccagtcttactgcccacccacctacagcaccactggctacagtgtggaccctgtggctggctaccagtacagccagtatggccaaactgctgttgattacctggccaaaaacgtgagcctgtccacacagcgccgtatgaagcttggggaacactccgctgtgctgggacttcttcctgtggaaacgggacaagcctactagggtccctggggcaacttgccccatccagtggcccagccaacccttcccaagccctgagtctcctcacctcagtccccttatccccctggggttgcaggagaccaagggaaaaaaaccctttcccttcctacaggaaaccctctggagacggaaaaccagtgtgccatctacccatgcttagtgacccagagtggccccttgccttcccctctttcttcagaggggttcctaggcatcctgcagtgacctccagctcacatccaccttctctgtatcgtggcctcggtcctgtctcagtgcagagattgaggctcaatttgaaccaagcacctagttatcagaagaaaatggtgccaaagacaaggccctggagtccttgacctctgagtcgtgggtgccctggctatgggtgtaggtggagcccatgggtgtcctcagtcacagagctgggagctctctctcgctcgcttggcatcaggactgcagcctctttcactggacactgagatgagtccccagggtgttcccaggggagaaagcaggtaacatcccagctttacctaggaatccagaggactttaggactgtccctatgcaccctgcagggcatcaggagaccaggaagggattctagcagagggtagggggcacagaggcagagctgattgcccatgggctatcccagaatgcctggtcctgaatctagcatcaggaggtgcaggattcctaggctgcaatctgacagaggcttgcccactgtgtcaggcctgggcagcccacagaacctgtcactctcctcaattggtaggagaagaggtcttgaggtgacaggaggcagaaggcaggctcagacagtcagagagcaccaagctttcaagtccgcacccctggggttcggcataccatcttgctggcagctggaaacctggttccctgaaagggggcctccatcctccagaatgtaaggctcttgatgccaccggatgcagagagccttctcgggccagacaaaattgctgctccaccccagagaagatgttccagccttcttggcatcttagaggaaggccatgtcgctgtccttttcagagtagcatatttttcagtgatggctgctcagtcaggaggcttctgtcgccttacaaagcacagtgcgctctgggcactgtttctaagccaccccatcccacccccacccccgccaccccggggacagaggaagatgctaaaagtcccagcaaagaggacaaagcacctttcttaagcactccagagtcttccttgtaccccgccctctcttagagctgggtcttttgagggaaacggattgctgagccctcccccccccccaatcctctcctctgtggagctgtttatcatcctctatttatcaaaatcgcatccatctttaccctctccttcgctatagcctacttctggatcaccctcatccagtgctggtaccccacagcactaaatccaggaaccctgggcttgatcacctgttgccacctgtacacatgaaaccacctgctggcccggcccatgtctcctgccctcagccagcaagacattcctagagagaggaactatgggcttaaaagccccaactgacttccttttgcctggggacctgaaccgacaagacaccagggacacttgtctacatgaacatgtgaccaatgtacaccgatttctcatctctagacctattatctgaagcctgtcccgggccatgactagaatggcttgtatctgtggtttagagaagtctaataataactgagggcaaactgactctctggtagcatggagcaccaggcggatggagctcaccagctctgtccaggtttcaaaggaggagactgttgggctcttcaaggtctggacaagaggaaagccacattgcccccttgggaacccaggttctccttttgaacttctcacagctgcaagcacccctttcaaagaccaaatgcatcctcctccacattccttgctccctggaggcctggctctggatacacctgagtcttcgttcacctactacactttaggagcaggaacttcaagcaggtgacatccacagggcccagtcccagccaagggagcaacattccaacgcttggaccaatcataatgatctgcccgtgagggtaaccgcaactagagacctgcttgggagaaaacaaaatgacttctcattccatgccatgcctctgaatgctcccccaagctgccatcttggtataaaatgggacttgtgttgtggggaaccccttgaccccaacaggttttcccaactgtctcatgcttttgtgaatctgtctgctttgatctgtaaaactcagccttgtttgggcagcttgtaatttcaacagtgaggcgacatcgattagatgagaggcaccaggcctctccgccgccgtccctctgtggccgtccctctggggttgagcagaacctagaagaaggccgatttccagtggccagactggaccagaaacagcccccaccccaatccctgtaaatagagtcaatagcaaaataagaggggcgccctccatgtcacctcaagtagctactggttcttctgtggaggcccctctgaactcattgtctggtagttgaaaatgtgatgttgtgctgtttgtttatagaacattggctttttatatataaatctatatacttaaaaacaaaaac

SEQ ID NO: 5 - protein, 320 aa, Homo sapiens
MELLSPPLRDVDLTAPDGSLCSFATTDDFYDDPCFDSPDLRFFEDLDPRLMHVGALLKPEEHSHFPAAVHPAPGAREDEHVRAPSGHHQAGRCLLWACKACKRKTTNADRRKAATMRERRRLSKVNEAFETLKRCTSSNPNQRLPKVEILRNAIRYIEGLQALLRDQDAAPPGAAAAFYAPGPLPPGRGGEHYSGDSDASSPRSNCSDGMMDYSGPPSGARRRNCYEGAYYNEAPSEPRPGKSAAVSSLDCLSSIVERISTESPAAPALLLADVPSESPPRRQEAAAPSEGESSGDPTQSPDAAPQCPAGANPNPIYQVL

SEQ ID NO: 6 - DNA, 1803 bp, Homo sapiens
aggggtgaggaagccctggggcgctgccgccgctttccttaaccacaaatcaggccggacaggagagggaggggtgggggacagtgggtgggcattcagactgccagcactttgctatctacagccggggctcccgagcggcagaaagttccggccactctctgccgcttgggttgggcgaagccaggaccgtgccgcgccaccgccaggatatggagctactgtcgccaccgctccgcgacgtagacctgacggcccccgacggctctctctgctcctttgccacaacggacgacttctatgacgacccgtgtttcgactccccggacctgcgcttcttcgaagacctggacccgcgcctgatgcacgtgggcgcgctcctgaaacccgaagagcactcgcacttccccgcggcggtgcacccggccccgggcgcacgtgaggacgagcatgtgcgcgcgcccagcgggcaccaccaggcgggccgctgcctactgtgggcctgcaaggcgtgcaagcgcaagaccaccaacgccgaccgccgcaaggccgccaccatgcgcgagcggcgccgcctgagcaaagtaaatgaggcctttgagacactcaagcgctgcacgtcgagcaatccaaaccagcggttgcccaaggtggagatcctgcgcaacgccatccgctatatcgagggcctgcaggctctgctgcgcgaccaggacgccgcgccccctggcgccgcagccgccttctatgcgccgggcccgctgcccccgggccgcggcggcgagcactacagcggcgactccgacgcgtccagcccgcgctccaactgctccgacggcatgatggactacagcggccccccgagcggcgcccggcggcggaactgctacgaaggcgcctactacaacgaggcgcccagcgaacccaggcccgggaagagtgcggcggtgtcgagcctagactgcctgtccagcatcgtggagcgcatctccaccgagagccctgcggcgcccgccctcctgctggcggacgtgccttctgagtcgcctccgcgcaggcaagaggctgccgcccccagcgagggagagagcagcggcgaccccacccagtcaccggacgccgccccgcagtgccctgcgggtgcgaaccccaacccgatataccaggtgctctgaggggatggtggccgcccacccgcccgagggatggtgcccctagggtccctcgcgcccaaaagattgaacttaaatgcccccctcccaacagcgctttaaaagcgacctctcttgaggtaggagaggcgggagaactgaagtttccgcccccgccccacagggcaaggacacagcgcggttttttccacgcagcacccttctcggagacccattgcgatggccgctccgtgttcctcggtgggccagagctgaaccttgaggggctaggttcagctttctcgcgccctcccccatgggggtgagaccctcgcagacctaagccctgccccgggatgcaccggttatttgggggggcgtgagacccagtgcactccggtcccaaatgtagcaggtgtaaccgtaacccacccccaacccgtttcccggttcaggaccactttttgtaatacttttgtaatctattcctgtaaataagagttgctttgccagagcaggagcccctggggctgtatttatctctgaggcatggtgtgtggtgctacagggaatttgtacgtttataccgcaggcgggcgagccgcgggcgctcgctcaggtgatcaaaataaaggcgctaatttataccgcc

SEQ ID NO: 7 - protein, 318 aa, Mus musculus
MELLSPPLRDIDLTGPDGSLCSFETADDFYDDPCFDSPDLRFFEDLDPRLVHMGALLKPEEHAHFPTAVHPGPGAREDEHVRAPSGHHQAGRCLLWACKACKRKTTNADRRKAATMRERRRLSKVNEAFETLKRCTSSNPNQRLPKVEILRNAIRYIEGLQALLRDQDAAPPGAAAFYAPGPLPPGRGSEHYSGDSDASSPRSNCSDGMMDYSGPPSGPRRQNGYDTAYYSEAARESRPGKSAAVSSLDCLSSIVERISTDSPAAPALLLADAPPESPPGPPEGASLSDTEQGTQTPSPDAAPQCPAGSNPNAIYQVL

SEQ ID NO: 8 - DNA, 1854 bp, Mus musculus
aggggccaggacgccccaggacacgactgctttcttcaccactcctctgacaggacaggacagggaggaggggtagaggacagccggtgtgcattccaacccacagaacctttgtcattgtactgttggggttccggagtggcagaaagttaagacgactctcacggcttgggttgaggctggacccaggaactgggatatggagcttctatcgccgccactccgggacatagacttgacaggccccgacggctctctctgctcctttgagacagcagacgacttctatgatgacccgtgtttcgactcaccagacctgcgcttttttgaggacctggacccgcgcctggtgcacatgggagccctcctgaaaccggaggagcacgcacacttccctactgcggtgcacccaggcccaggcgctcgtgaggatgagcatgtgcgcgcgcccagcgggcaccaccaggcgggtcgctgcttgctgtgggcctgcaaggcgtgcaagcgcaagaccaccaacgctgatcgccgcaaggccgccaccatgcgcgagcgccgccgcctgagcaaagtgaatgaggccttcgagacgctcaagcgctgcacgtccagcaacccgaaccagcggctacccaaggtggagatcctgcgcaacgccatccgctacatcgaaggtctgcaggctctgctgcgcgaccaggacgccgcgccccctggcgccgctgccttctacgcacctggaccgctgcccccaggccgtggcagcgagcactacagtggcgactcagatgcatccagcccgcgctccaactgctctgatggcatgatggattacagcggccccccaagcggcccccggcggcagaatggctacgacaccgcctactacagtgaggcggcgcgcgagtccaggccagggaagagtgcggctgtgtcgagcctcgactgcctgtccagcatagtggagcgcatctccacagacagccccgctgcgcctgcgctgcttttggcagatgcaccaccagagtcgcctccgggtccgccagagggggcatccctaagcgacacagaacagggaacccagaccccgtctcccgacgccgcccctcagtgtcctgcaggctcaaaccccaatgcgatttatcaggtgctttgagagatcgactgcagcagcagagggcgcaccaccgtaggcactcctggggatggtgtccctggttcttcacgcccaaaagatgaagcttaaatgacactcttcccaactgtcctttcgaagccgttcttccagagggaagggaagagcagaagtctgtcctagatccagccccaaagaaaggacatagtcctttttgttgttgttgttgtagtccttcagttgtttgtttgttttttcatgcggctcacagcgaaggccacttgcactctggctgcacctcactgggccagagctgatccttgagtggccaggcgctcttcctttcctcatagcacaggggtgagccttgcacacctaagccctgccctccacatccttttgtttgtcactttctggagccctcctggcacccacttttccccacagcttgcggaggccactcaggtctcaggtgtaacaggtgtaaccataccccactctcccccttcccgcggttcaggaccacttatttttttatataagacttttgtaatctattcgtgtaaataagagttgcttggccagagcgggagccccttgggctatatttatctcccaggcatgctgtgtagtgcaacaaaaactttgtatgtttattcctcaagcgggcgagcctcgaggctcgctcgctcaggtgttggaaataaagacgctaattt

(19) United States
(12) Patent Application Publication
(10) Pub. No.: US 2022/0041990 A1
(43) Pub. Date: Feb. 10, 2022

(54) METHODS, COMPOSITIONS, AND KITS FOR PRODUCING SKELETAL MUSCLE STEM CELLS AND TREATING DISORDERS

(71) Applicant: The Johns Hopkins University, Baltimore, MD (US)

(72) Inventors: Gabsang Lee, Baltimore, MD (US); Kathryn Wagner, Baltimore, MD (US); Congshan Sun, Baltimore, MD (US); InYoung Choi, Baltimore, MD (US); Ho Tae Lim, Baltimore, MD (US); Peter Andersen, Battimore, MD (US)

(21) Appl. No.: 17/599,431

(22) PCT Filed: Apr. 3, 2020
(86) PCT No.: PCT/US2020/026603

§ 371 Date: Sep. 28, 2021

Related U.S. Application Data
(60) Provisional application No. 62/828,973, filed on Apr. 3, 2019.

(51) Int. Cl. C12N 5/077; A61K 35/34; A61P 21/00

(52) U.S. Cl. CPC C12N 5/0658; A61P 21/00; A61K 35/34

(57) ABSTRACT

Provided herein are, inter alia, are methods, compositions and kits for producing populations of myogenic progenitor cells, and compositions comprising the cell populations derived thereof. Also included are methods and compositions for treating or preventing a muscle disease or disorder in a subject (e.g., Duchenne muscular dystrophy (DMD).

### CROSS REFERENCE TO RELATED APPLICATIONS

This application claims the benefit of U.S. Provisional Application 62/828,973 filed on Apr. 3, 2019. The entire contents of this application are incorporated herein by reference in their entirety.

### GOVERNMENT SUPPORT

This invention was made with government support under Grant No. R01AR070751 awarded by the National Institute of Health (NIH). The government has certain rights in the invention.

### FIELD

The present disclosure is directed to skeletal muscle cell replacement therapy which includes producing populations of myogenic progenitor cells.

### BACKGROUND

A major roadblock in skeletal muscle cell replacement therapy is limited integration of transplanted skeletal muscle cells at the injury site. Most cell transplantations require multiple cell injections because patients need new cells continuously in their live. Thus, new methods for producing therapies that target muscle diseases and syndromes are needed.

### SUMMARY

Provided herein are, inter alia, methods, compositions and kits for producing populations of myogenic progenitor cells as well as the progeny derived thereof. Also included are methods and compositions for treating or preventing a muscle diseases or disorder in a subject (e.g., Duchenne muscular dystrophy (DMD)).

It is demonstrated herein, for the first time, that myogenic progenitor cells derived from pluripotent stem cells in vitro can reside in the muscle stem cell niche upon intra-muscular transplantation, become quiescent and change their molecular expression profile that is similar to human muscle stem cells. Such myogenic progenitor cells, therefore, can be used for therapeutic purposes, such as treating a degenerative muscle wasting disease or condition. In one embodiment, therefore, provided is a population of mammalian cells, wherein at least 30% of the cells are PAX7+ myogenic progenitor cells (MPCs) derived from pluripotent stem cells in vitro.

In some embodiments, the PAX7+ MPCs express one or more of CHRNA1 (Cholinergic receptor nicotinic alpha 1), NTSR1 (Neurotensin receptor 1), or FZD1 (Frizzled class receptor 1), and does not express one or more of FZD5 (Frizzled class receptor 5), GPR37 (G protein-coupled receptor 37), or GPR27 (G protein-coupled receptor 27).

In some embodiments, the pluripotent stem cells are induced pluripotent stem cells (iPSC) or embryonic stem cells (ESC), in particular human pluripotent stem cells.

Another embodiment provides a method for treating a degenerative muscle wasting disease or condition in a patient in need thereof. In some embodiments, the method entails injecting to the patient the cell population of the present disclosure. Example degenerative muscle wasting diseases or conditions include muscular dystrophy (such as Duchenne muscular dystrophy (DMD)), myopathy, a mitochondrial disease, soft tissue sarcoma, an ion channel disease, cachexia and sarcopenia.

Also provided is a method for producing a population of myogenic progenitor cells (MPCs), the method comprising: differentiating a plurality of pluripotent stem cell in a medium comprising a selective inhibitor of glycogen synthase kinase 3 (GSK-3) to obtain differentiated cells; treating the differentiated cells with an inhibitor of Notch signaling; and expanding the differentiated cells with a fibroblast growth factor (FGF), thereby obtaining a population of MPCs expressing PAX7 (paired box protein).

In addition, provided herein are methods for producing a population of myogenic progenitor cells (MPCs). In aspects, the method comprises obtaining a cell population from a subject, producing a pluripotent stem cell (PSC) population from the cell population, wherein the pluripotent stem cell population is an embryonic stem cell (ESC) population; and producing the myogenic progenitor cell population from the pluripotent stem cell population.

In embodiments, the methods herein provide that the pluripotent stem cell population is cultured in a cell culture medium comprising basic fibroblast growth factor (FGF2) and/or fibroblast growth factor 8.

In embodiments, the myogenic progenitor cell of the methods herein express myogenic markers, the myogenic markers comprising paired box protein (PAX7) or MyoD. In embodiments, the myogenic progenitor cells express green fluorescent protein (GFP).

In other embodiments, the methods provided herein produce a cell population effective to increase PAX7 expression in cells of the myogenic progenitor cell population.

In embodiments, the methods described herein produce a pluripotent stem cell population that is cultured and expanded ex vivo, e.g., for at least about 30 days. In other embodiments, the pluripotent stem cell population is cultured ex vivo for about 5 days, about 10 days, about 20 days, about 30 days, about 40 days, about 50 days, about 60 days or more.

In other embodiments, the pluripotent stem cell population is cultured ex vivo, to expand their number.

In embodiments, the methods herein provide that the pluripotent stem cell population is produced from a cell population obtained from the subject.

In other embodiments, the pluripotent stem cell population produced by the methods described herein is an induced pluripotent stem (iPS) cell population.

In additional embodiments, the cell population is obtained from a subject (e.g., a human subject). In further embodiments, the cell population is obtained via a biopsy.

In certain aspects, a population of myogenic progenitor cells produced according to the methods described herein are used to treat or prevent a muscle disease or disorder (e.g., in a subject in need thereof). In aspects, the method further comprises administering to the subject an effective amount of the population of myogenic progenitor cells produced according to the methods described herein.

In embodiments, the muscle disease comprises Duchenne muscular dystrophy (DMD). In other embodiments, the muscle disorder comprises a muscle wasting disorder, for example, the muscle wasting disorder may include cachexia.

In embodiments, the cells are isolated before the muscle disease begins in the subject. In other embodiments, the cells are isolated after the muscle disease begins in the subject.

In embodiments the pluripotent stem cell population produced by the methods here in is cultured to expand their number before being administered to the subject.

In embodiments, provided herein are methods for increasing the level of an early myogenic marker in a subject in need thereof, the method comprising administering to the subject, an effective amount of the population of myogenic progenitor cells produced according to the methods described herein.

Also provided herein are kits for producing myogenic progenitor cells, the kit comprising a cell culture media or a cell culture medium, wherein the cell culture medium is suitable for culturing a pluripotent stem cell (PSC) population. In embodiments, the kits described herein include a cell culture medium is a serum-free cell culture medium.

### DESCRIPTION OF THE DRAWINGS

The patent or application file contains at least one drawing executed in color. Copies of this patent or patent application publication with color drawings will be provided by the Office upon request and payment of the necessary fee.

FIGS. 1A-1E show that PAX7::MPC derived from human pluripotent stem cells (hPSCs) could be maintained ex vivo and participate in muscle regeneration. FIG. 1A is a schematic showing a scheme of myogenic differentiation from PAX7::GFP hPSC to PAX7::GFP MPCs. FIG. 1B depicts flow cytometry analysis of PAX7::GFP human embryonic stem cell (hESC) after 30 days of myogenic differentiation, H9 ESC derived myoblasts (no GFP control) and PAX7GFP MPC after being passaged twice. The GFP expressing cells were gated. FIG. 1C depicts a schematic showing a cell transplantation scheme. FIG. 1D depicts representative images of cross section of TAs at 4-week post sorted and expanded or unsorted cell from PAX7::GFP hESCs myogenic linage differentiation culture showing huDYSTROPHIN positive fibers and human cells labeled by huLAMINA/C nuclei. FIG. 1E is a bar graph showing the quantification of huDYSTROPHIN fibers positive area among the entire TA area.

FIGS. 2A-2G show that PAX7::GFP progenies can be isolated and mimic the quiescent muscle stem cells on the cellular level. FIG. 2A depict images of a FACs plot of PAX7::GFP⁺ cell isolation 4 weeks after either unsorted or sorted and expanded cell transplantation. TA injected with vector medium was used by control. FIG. 2B depicts a graph of the quantification of % of PAX7::GFP cells among all the isolated mononucleated cells from single mice (n(Control)=4, n(Sorted & Expanded)=14, n(Unsorted)=8). FIGS. 2C and 2D depict graphs showing single cell Q PCR of cells isolated from PAX7::GFP cells following myogenic differentiation, before and post expansion (B.E and P.E) as well as 4 weeks post transplantation (in vivo). FIG. 2E depicts images 4 weeks after the PAX7::GFP MPC transplantation, TA were sections and PAX7::GFP MPC progenies could be identified by double labeling of huLAMINA/C and PAX7 protein. They were located under basal Lamina (yellow arrow). FIG. 2F depict Pyronin Y and Hoescht cell cycle analysis of PAX7::MPCs in vivo as well as in ex vivo culture showing PAX7::MPCs in vivo have low DNA and RNA content. FIG. 2G is a pie graph that shows the percentage of cells at different cell cycle from Pax7::GFP in vivo and PAX7::GFP MPC ex vivo.

FIGS. 3A-3F show single-cell RNA sequencing of PAX7::GFP⁺ MPC in vivo compared with cells before transplantation. Cell by isolated by GFP signal. FIG. 3A depicts graph of RNA content of PAX7::GFP⁺ MPC in vivo, ex vivo before transplantation, ex vivo cultured for 1 month and OCT4::GFP⁺ hESCs. FIG. 3B depicts a PCA plot that shows the distinctive expression RNA expression profiles between PAX7::GFP⁺ MPC in vitro, ex vivo and OCT4::GFP⁺ hESCs. However, the one month ex vivo culture did not change the gene expression profile. FIG. 3C depicts a volcano plot showing that most genes were down-regulated and some genes were upregulated compared between PAX7::GFP MPC in vivo and ex vivo before transplantation. FIG. 3D depicts an image of up- and down-regulated genes clustered by function. FIGS. 3E and 3F depict images of a cluster analysis showing gene ontology enrichment of up- and down-regulated genes.

FIGS. 4A-4E show images of reinjury and serial transplantation of PAX7::GFP MPCs. FIG. 4A depicts a scheme of reinjury experiment. FIG. 4B depicts images showing 4 weeks post the second injury, TA were sectioned and stained with huDYSTROPHIN and LAMINA/C to measure the participation of muscle regeneration of engrafted PAX7::MPCs. FIG. 4C depicts a graph of the quantification of huDYSTROPHIN positive areas among the entire TA area 4 weeks post the second injury. FIG. 4D depicts a scheme of serial transplantation. FIG. 4E depicts images showing that PAX7::GFP⁺ MPCs were isolated from the 1st recipient mouse and transplanted into the 2nd recipient mouse. The TA of the 2nd recipient mouse were sectioned and PAX7 and huLAMINA/C were used to trace transplanted cells. The co-labeled cells were found to reside under basal lamina (arrow).

FIGS. 5A-5D depict the characterization of PAX7::GFP MPCs. FIG. 5A is an illustration of generation of PAX7::GFP hESCs by CRISPR Cas9. FIG. 5B is a bar graph showing the relative gene expression of PAX7, MYOD, GFP and NEUROGENIN from PAX7::GFP⁺ MPCs post expansion (P.E), before expansion (B.E.) and non-myoblast cells.

FIG. 5C are images depicting immunocytochemistry picture of PAX7, MYOD and GFP protein expression in the PAX7::GFP MPC ex vivo expansion culture. FIG. 5D are images showing that PAX7::GFP MPCs could form myotubes labeled by MyHC and DESMIN antibodies under differentiation condition.

FIG. 6 is a graph showing the results from the transplantation studies.

FIG. 7 is a graph showing the results from a treadmill study in the in vivo model of Duchenne's after transplantation into the mice.

FIGS. 8A and 8B show a blot and a series of graphs demonstrating the human origin of the PAX7::GFP⁺ cells post-transplantation. FIG. 8A: genomic PCR was conducted using human specific primers to determine the origin of PAX7::GFP⁺ cells isolated from injected TA. FIG. 8B: Sequence comparison of single cell RNA-seq result shows that the majority of the PAX7::GFP⁺ cells isolated from injected TA are human cells.

FIG. 9 is a plot demonstrating that the transplanted cells become quiescent cells after 4 weeks post-transplantation in vivo. The results are shown as: 1 week after injection into mice; 2 weeks after injection into mice; 3 weeks after injection into mice; and 4 weeks after injection into mice. PAX7::GFP⁺ cells were collected at 1-week, 2-week, 3-week and 4-week post transplantation. From the SNE plot it can be seen that from week 1 post-transplantation, the transcriptome of PAX7::GFP⁺ cells does not change significantly with time.

### DETAILED DESCRIPTION

Provided herein are, inter alia, methods, compositions and kits for producing populations of myogenic progenitor cells, and the prepared myogenic progenitor cells. Also included are methods and compositions for treating or preventing a muscle diseases or disorder in a subject (e.g., Duchenne muscylar dystrophy (DMD)). Also provided herein are methods and compositions for treatment of wasting disorders, cancer, patients with limited mobility, and athletes.

In aspects, provided herein are methods of treating a subject with a muscle wasting disorder, heart disease, exercise-induced muscle weakness, or cancer. In aspects, applications may include diseases or conditions wherein improvement in skeletal muscle strength may be beneficial. In certain aspects, diseases or conditions may include muscle wasting due to AIDS, age-related Sarcopenia, cancer cachexia, Cushing's syndrome, diabetes mellitus and sepsis. Additionally, muscle strength improvement in patients suffering from amytrophic lateral sclerosis (ALS) and Duchenne muscular dystrophy (DMD) is contemplated. In further aspects, methods provided herein can be used in healthy patients, for example to increase physical exertion such as increasing their walk or run speed.

A major roadblock in skeletal muscle cell replacement therapy is the limited integration of transplanted skeletal muscle cells at the injury site. Although transplantation of mouse skeletal muscle stem cells improves motor function, little is known about whether human embryonic PAX7⁺ cells can be functional and serially re-populated in postnatal in vivo environments, transitioning their cellular maturation stage.

Provided herein is a novel approach to generating human PAX7::GFP⁺ cells that can survive as quiescent and functional local skeletal muscle stem cells in niche area of in vivo environments. In this study, hPSCs were directly differentiated into myogenic linages and isolated PAX7::GFP expressing myogenic progenitor cells (PAX7::GFP MPCs).

Also provided, is that these cells could be maintained and expanded ex vivo. When transplanted in vivo, they could participate in the muscle regeneration by fusing into muscle fiber as well as becoming mononucleated PAX7 expressing cells residing under basal lamina. The progenies of PAX7::GFP MPCs were traced and isolated with GFP expression. Together with single cell RNA sequence and cellular measurement, the unique quiescent muscle stem cell gene characteristics was observed from these cells. Reinjury and serial transplantation experiments further confirmed their self-renewal and regeneration capability. This study demonstrated the transition from hPSC derived myogenic progenitor cells to quiescent muscle stem cells in vivo with single cell experimental approach.

### General Definitions

The following definitions are included for the purpose of understanding the present subject matter and for constructing the appended patent claims. The abbreviations used herein have their conventional meanings within the chemical and biological arts.

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

Unless defined otherwise, technical and scientific terms used herein have the same meaning as commonly understood by a person of ordinary skill in the art. See, e.g., Singleton et al., DICTIONARY OF MICROBIOLOGY AND MOLECULAR BIOLOGY 2nd ed., J. Wiley & Sons (New York, N.Y. 1994); Sambrook et al., MOLECULAR CLONING, A LABORATORY MANUAL, Cold Springs Harbor Press (Cold Springs Harbor, NY 1989). Any methods, devices and materials similar or equivalent to those described herein can be used in the practice of this invention. The following definitions are provided to facilitate understanding of certain terms used frequently herein and are not meant to limit the scope of the present disclosure.

In embodiments, a “muscle disease” or “muscle syndrome” or “muscle condition” or “myopathy” is a disorder that results in increasing weakening and breakdown of skeletal muscles over time. For example, muscular dystrophy (MD) contains at least thirty different genetic disorders that are usually classified into nine main categories or types. MD refers to a group of hereditary, progressive, degenerative disorders characterized by progressive muscle weakness, defects in muscle proteins, and the destruction of muscle fibers and tissue over time. In many cases, the histological picture shows variation in fiber size, muscle cell necrosis and regeneration, and often proliferation of connective and adipose tissue. The diseases primarily target the skeletal or voluntary muscles. However, muscles of the heart and other involuntary muscles are also affected in certain forms of muscular dystrophy.

The most common type of MD is Duchenne muscular dystrophy (DMD) which typically affects males beginning around the age of four. Other types include Becker muscular dystrophy, facioscapulohumeral muscular dystrophy, and myotonic dystrophy. They are due to mutations in genes that are involved in making muscle proteins. This can occur due to either inheriting the defect from one's parents or the mutation occurring during early development. Disorders may be X-linked recessive, autosomal recessive, or autosomal dominant. Additional examples of muscular dystrophies include Duchenne Muscular Dystrophy, Becker Muscular Dystrophy, Limb Girdle Muscular Dystrophy, Facioscapulohumeral Muscular Dystrophy, Oculopharyngeal muscular dystrophy, Emery-Dreifuss muscular dystrophy, Fukuyama-type congenital muscular dystrophy, Miyoshi myopathy, Ullrich congenital muscular dystrophy, Steinert Muscular Dystrophy.

Duchenne Muscular Dystrophy (DMD) is the most common inherited lethal childhood muscular dystrophy, affecting about 1 in 3000 males. Children with DMD usually become wheelchair bound by the age of 11 or 12 years and affected individuals usually die in the second or third decade of life. DMD originates from mutations in the dystrophin gene located on the X chromosome (Xp21), leading to loss of dystrophin protein with attendant muscle fiber destruction. Although the role of the dystrophin protein in maintaining skeletal myofiber integrity is generally well recognized, the exact mechanism that leads to myofiber destruction and loss in dystrophic muscle is not well understood. The discovery of the dystrophin gene and the subsequent characterization of the protein product have established dystrophin as an integral sarcolemmal protein, linking the muscle sarcomere and cytoskeleton to the surrounding extracellular matrix. The localization of dystrophin is synonymous with maintaining muscle integrity and its absence (as evidenced in DMD) leads to membrane fragility, contraction induced myofiber damage, and death.

### Current DMD Treatment

There is no known cure for DMD, and an ongoing medical need has been recognized by regulatory authorities. Treatment is generally aimed at controlling the onset of symptoms to maximize the quality of life which can be measured using specific questionnaires, and include:
Corticosteroids such as prednisolone and deflazacort lead to short-term improvements in muscle strength and function up to 2 years. Corticosteroids have also been reported to help prolong walking.
β₂ agonists that increase muscle strength, but do not modify disease progression (e.g., salbutamol (e.g., albuterol) which is a β₂ agonist may be used).
Mild, nonjarring physical activity such as swimming is encouraged. Inactivity (such as bed rest) can worsen the muscle disease.
Physical therapy is helpful to maintain muscle strength, flexibility, and function.
Orthopedic appliances (such as braces and wheelchairs) may improve mobility and the ability for self-care. Form-fitting removable leg braces that hold the ankle in place during sleep can defer the onset of contractures.
Appropriate respiratory support as the disease progresses is important.
Cardiac problems may require a pacemaker.
The medication, e.g., ataluren may also be provided.

As used herein, a “wasting syndrome” is a disease or condition which results in, is characterized by or accompanied by a loss of muscle mass and/or strength. Examples of such diseases include AIDS; cancer; demyelinating disorders resulting in muscle atrophy (e.g., multiple sclerosis, amyotropic lateral sclerosis, congenital metabolic disorders such as phenylketonuria, Tay-Sachs disease, Hurler's syndrome and leukodystrophies, postinfections encephalomyelitis, viral encephalitis, aseptic meningitis and HTLV-associated myelopathy); dystrophic disease (e.g., muscular dystrophy, Duchenne dystrophy, Landouzy-Dejerine muscular dystrophy, and limb-girdle muscular dystrophy); generalized and focal dystonia; eating disorders (e.g., anorexia and bulimia); cachexia or wasting due to chronic diseases; and vascular disorders (e.g., infarction). Loss of muscle mass and/or strength can also occur in subjects undergoing certain types of chemotherapy, or as a consequence of aging, malnutrition, or muscle deconditioning. Muscle deconditioning commonly occurs in individuals who experience a prolonged period in a weightless environment such as outer space, are bedridden for extended period of time, or have certain muscles or muscle groups immobilized, such as in a cast. Individuals requiring prolonged bedrest include those with chronic diseases and those suffering from temporary paralysis from spinal cord injuries resulting from, for example, hematoma or compression. In embodiments, the wasting disorder is cachexia (e.g., chemotherapy-induced cachexia), aging-related cachexia or sports medicine related.

“Cachexia” is weakness and a loss of weight caused by a disease or as a side effect of illness. Cardiac cachexia, i.e. a muscle protein wasting of both the cardiac and skeletal muscle, is a characteristic of congestive heart failure. Cancer cachexia is a syndrome that occurs in patients with solid tumors and hematological malignancies and is manifested by weight loss with massive depletion of both adipose tissue and lean muscle mass. Acquired Immunodeficiency Syndrome (AIDS). Cachexia is a Human Immunodeficiency Virus (HIV) associated myopathy and/or muscle weakness/wasting that is a relatively common clinical manifestation of AIDS. Individuals with HIV-associated myopathy or muscle weakness or wasting typically experience significant weight loss, generalized or proximal muscle weakness, tenderness, and muscle atrophy.

As used herein, “sample,” “patient sample,” “biological sample,” and the like, encompass a variety of sample types obtained from a patient, individual, or subject and can be used in a diagnostic, prognostic and/or monitoring assay.” The biological samples used in the present invention can include cells, protein or membrane extracts of cells, blood or biological fluids such as ascites fluid or brain fluid (e.g., cerebrospinal fluid). Examples of solid biological samples include, but are not limited to, samples taken from tissues of the central nervous system, bone, breast, kidney, cervix, endometrium, head/neck, gallbladder, parotid gland, prostate, pituitary gland, muscle, esophagus, stomach, small intestine, colon, liver, spleen, pancreas, thyroid, heart, lung, bladder, adipose, lymph node, uterus, ovary, adrenal gland, testes, tonsils, thymus and skin, or samples taken from tumors. Examples of “body fluid samples” include, but are not limited to blood, serum, semen, prostate fluid, seminal fluid, urine, feces, saliva, sputum, mucus, bone marrow, lymph, and tears.

The patient sample may be obtained from a healthy subject, a diseased patient or a patient having associated symptoms of a muscle diseases or disorder in a subject (e.g., Duchenne muscular dystrophy (DMD)). In particular embodiments, a “sample” (e.g., a test sample) from a subject refers to a sample that might be expected to contain elevated levels of the protein markers of the invention in a subject having a muscle diseases or disorder in a subject. In certain embodiments, a sample that is “provided” can be obtained by the person (or machine) conducting the assay, or it can have been obtained by another, and transferred to the person (or machine) carrying out the assay.

In certain embodiments, a biological sample is obtained from one or more sources comprising: autologous, allogeneic, haplotype matched, haplotype mismatched, haplo-identical, xenogeneic, cell lines or combinations thereof.

Moreover, a sample obtained from a patient can be divided and only a portion may be used for diagnosis. Further, the sample, or a portion thereof, can be stored under conditions to maintain sample for later analysis. The definition specifically encompasses blood and other liquid samples of biological origin (including, but not limited to, peripheral blood, serum, plasma, cord blood, amniotic fluid, cerebrospinal fluid, urine, saliva, stool and synovial fluid), solid tissue samples such as a biopsy specimen or tissue cultures or cells derived therefrom and the progeny thereof. In certain embodiments, a sample comprises a bone marrow or blood sample.

The definition of “sample” also includes samples that have been manipulated in any way after their procurement, such as by centrifugation, filtration, precipitation, dialysis, chromatography, treatment with reagents, washed, or enriched for certain cell populations. The terms further encompass a clinical sample, and also include cells in culture, cell supernatants, tissue samples, organs, and the like. Samples may also comprise fresh-frozen and/or formalin-fixed, paraffin-embedded tissue blocks, such as blocks prepared from clinical or pathological biopsies, prepared for pathological analysis or study by immunohistochemistry.

A “marker” as used herein is used to describe the characteristics and/or phenotype of a cell. Markers can be used for selection of cells comprising characteristics of interests. Markers will vary with specific cells. Markers are characteristics, whether morphological, functional or biochemical (enzymatic) characteristics of the cell of a particular cell type, or molecules expressed by the cell type. Preferably, such markers are proteins, and more preferably, possess an epitope for antibodies or other binding molecules available in the art. However, a marker may consist of any molecule found in a cell including, but not limited to, proteins (peptides and polypeptides), lipids, polysaccharides, nucleic acids and steroids. Examples of morphological characteristics or traits include, but are not limited to, shape, size, and nuclear to cytoplasmic ratio. Examples of functional characteristics or traits include, but are not limited to, the ability to adhere to particular substrates, ability to incorporate or exclude particular dyes, ability to migrate under particular conditions, and the ability to differentiate or dedifferentiate along particular lineages. Markers may be detected by any method available to one of skill in the art. Markers can also be the absence of a morphological characteristic or absence of proteins, lipids etc. Markers can be a combination of a panel of unique characteristics of the presence and absence of polypeptides and other morphological characteristics.

In embodiments, the muscle disorder is “sarcopenia.” Sarcopenia is a debilitating disease that afflicts the elderly and chronically ill patients and is characterized by loss of muscle mass and function. It is established that anabolic steroids can prevent and/or reverse losses in lean body mass (decrease in skeletal muscle mass) associated with age, disease and trauma injury. Further, increased lean body mass is associated with decreased morbidity and mortality for certain muscle-wasting disorders.

The terms “muscle wasting” or “muscular wasting”, used herein interchangeably, refer to the progressive loss of muscle mass and/or to the progressive weakening and degeneration of muscles, including the skeletal or voluntary muscles which control movement, cardiac muscles which control the heart, and smooth muscles.

Muscular atrophy, as used herein, refers to a partial or complete loss of muscle mass. Muscle dystrophy is a muscle disease involving progressive muscle weakness and atrophy and death of muscle cells and tissues. Muscle atrophy may include diseases or conditions accompanied by, for example, muscle weakness accompanied by muscle atrophy, in particular, a decrease in muscle mass or muscle weakness of proximal muscles, a decrease in muscle function, a decrease of muscle mass, etc. Muscular atrophy or muscle dystrophy may be muscular atrophy caused by long-term bed rest, muscular atrophy caused by an assistive device for therapy, or muscular atrophy caused by cachexia, amyotrophic lateral sclerosis, spinal progressive muscular atrophy, muscular dystrophy, or a combination thereof.

By “muscle stem cell” is meant a self-renewing mononucleate cell that produces as progeny mononucleate myoblasts, which are committed to form multinucleate myofibers via intercellular fusion. Encompassed herein, are muscle stem cells that produce skeletal muscle, smooth muscle, or cardiac muscle.

The term “muscle cell” as used herein refers to any cell which contributes to muscle tissue. Myoblasts, satellite cells, myotubes, and myofibril tissues are all included in the term “muscle cells” and may all be treated using the methods of the invention. Muscle cell effects may be induced within skeletal, cardiac and smooth muscles. Muscle tissue in adult vertebrates will regenerate from reserve myoblasts called “satellite cells”. Satellite cells are distributed throughout muscle tissue and are mitotically quiescent in the absence of injury or disease. Following muscle injury or during recovery from disease, satellite cells will reenter the cell cycle, proliferate and 1) enter existing muscle fibers or 2) undergo differentiation into multinucleate myotubes which form new muscle fiber. The myoblasts ultimately yield replacement muscle fibers or fuse into existing muscle fibers, thereby increasing fiber girth by the synthesis of contractile apparatus components. This process is illustrated, for example, by the nearly complete regeneration which occurs in mammals following induced muscle fiber degeneration; the muscle progenitor cells proliferate and fuse together regenerating muscle fibers.

“Muscle growth” as used herein refers to the growth of muscle which may occur by an increase in the fiber size and/or by increasing the number of fibers. The growth of muscle as used herein may be measured by A) an increase in wet weight, B) an increase in protein content, C) an increase in the number of muscle fibers, or D) an increase in muscle fiber diameter. An increase in growth of a muscle fiber can be defined as an increase in the diameter where the diameter is defined as the minor axis of ellipsis of the cross section.

“Myogenic” cells as described herein are those cells that are related to the origin of muscle cells or fibers. Various molecular markers are known to be specific for the middle and late stages of myogenic differentiation. For example, in C2C12 cells, myosin and MRF4 mark the late stages of myogenesis and are largely restricted to myotubes, whereas myogenin and nestin mark the middle stages of myogenesis and are found in all myotubes and in many committed myoblasts.

As used herein “satellite cells,” or “myosatellite cells,” refers to small multipotent cells with little cytoplasm found in mature muscle. Satellite cells are precursors to skeletal muscle cells, able to give rise to satellite cells or myoblasts, which give rise to skeletal muscle cells. They have the potential to provide additional myonuclei to their parent muscle fiber, or return to a quiescent state. Upon activation, satellite cells can re-enter the cell cycle to proliferate and differentiate into myoblasts. Satellite cells may exhibit one or more features which may be shared with endogenous satellite cells, including, but not limited to, capacity to repopulate the satellite cell niche, ability to drive muscle regeneration, exhibit appropriate expression of gene markers, appropriate expression of glycoproteins, and expandability in culture.

“Atrophy” or “wasting” of muscle as used herein refers to a significant loss in muscle fiber girth. By significant atrophy is meant a reduction of muscle fiber diameter in diseased, injured or unused muscle tissue of at least 10% relative to undiseased, uninjured, or normally utilized tissue.

The term “disease” refers to any deviation from the normal health of a mammal and includes a state when disease symptoms are present, as well as conditions in which a deviation (e.g., muscle dysfunction or muscle disorder) has occurred, but symptoms are not yet manifested.

“Patient” or “subject in need thereof” refers to a living member of the animal kingdom suffering from or who may suffer from the indicated disorder. In embodiments, the subject is a member of a species comprising individuals who may naturally suffer from the disease. In embodiments, the subject is a mammal. Non-limiting examples of mammals include rodents (e.g., mice and rats), primates (e.g., lemurs, bushbabies, monkeys, apes, and humans), rabbits, dogs (e.g., companion dogs, service dogs, or work dogs such as police dogs, military dogs, race dogs, or show dogs), horses (such as race horses and work horses), cats (e.g., domesticated cats), livestock (such as pigs, bovines, donkeys, mules, bison, goats, camels, and sheep), and deer. In embodiments, the subject is a human.

The terms “subject,” “patient,” “individual,” etc. are not intended to be limiting and can be generally interchanged. That is, an individual described as a “patient” does not necessarily have a given disease, but may be merely seeking medical advice.

The transitional term “comprising,” which is synonymous with “including,” “containing,” or “characterized by,” is inclusive or open-ended and does not exclude additional, unrecited elements or method steps. By contrast, the transitional phrase “consisting of” excludes any element, step, or ingredient not specified in the claim. The transitional phrase “consisting essentially of” limits the scope of a claim to the specified materials or steps “and those that do not materially affect the basic and novel characteristic(s)” of the claimed invention.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

It is understood that where a parameter range is provided, all integers within that range, and tenths thereof, are also provided by the invention. For example, “0.2-5 mg” is a disclosure of 0.2 mg, 0.3 mg, 0.4 mg, 0.5 mg, 0.6 mg etc. up to and including 5.0 mg.

As used in the description herein and throughout the claims that follow, the meaning of “a,” “an,” and “the” includes plural reference unless the context clearly dictates otherwise.

As used herein, “treating” or “treatment” of a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of development of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean inhibiting the progression of the condition, disorder or disease, slowing the progression of the condition, disorder or disease temporarily, although in some instances, it involves halting the progression of the condition, disorder or disease permanently.

As used herein, the terms “treat” and “prevent” are not intended to be absolute terms. In embodiments, treatment can refer to a 10%, 20%, 30%, 40%, 50%, 60%, 70%, 80%, 90%, or 100% reduction in the severity of an established disease, condition, or symptom of the disease or condition. In embodiments, a method for treating a disease is considered to be a treatment if there is a 10% reduction in one or more symptoms of the disease in a subject as compared to a control. Thus the reduction can be a 10%, 20%, 30%, 40%, 50%, 60%, 70%, 80%, 90%, 100%, or any percent reduction in between 10% and 100% as compared to native or control levels. It is understood that treatment does not necessarily refer to a cure or complete ablation of the disease, condition, or symptoms of the disease or condition. In embodiments, references to decreasing, reducing, or inhibiting include a change of 10%, 20%, 30%, 40%, 50%, 60%, 70%, 80%, 90% or greater as compared to a control level and such terms can include but do not necessarily include complete elimination. In embodiments, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques.

The terms “effective amount,” “effective dose,” etc. refer to the amount of an agent that is sufficient to achieve a desired effect, as described herein. In embodiments, the term “effective” when referring to an amount of cells or a therapeutic compound may refer to a quantity of the cells or the compound that is sufficient to yield an improvement or a desired therapeutic response without undue adverse side effects (such as toxicity, irritation, or allergic response) commensurate with a reasonable benefit/risk ratio when used in the manner of this disclosure. In embodiments, the term “effective” when referring to the generation of a desired cell population may refer to an amount of one or more compounds that is sufficient to result in or promote the production of members of the desired cell population, especially compared to culture conditions that lack the one or more compounds.

### Myogenesis and Skeletal Muscle

Myogenesis is the formation of muscular tissue, e.g., particularly during embryonic development. Muscle fibers generally form the fusion of myoblasts into multi-nucleated fibers called myotubes. In the early development of an embryo, myoblasts can either proliferate, or differentiate into a myotube. What controls this choice in vivo is generally unclear. If placed in cell culture, most myoblasts will proliferate if enough fibroblast growth factor (FGF) or another growth factor is present in the medium surrounding the cells. When the growth factor runs out, the myoblasts cease division and undergo terminal differentiation into myotubes. Myoblast differentiation proceeds in stages. The first stage, involves cell cycle exit and the commencement of expression of certain genes. The second stage of differentiation involves the alignment of the myoblasts with one another. Studies have shown that even rat and chick myoblasts can recognize and align with one another, suggesting evolutionary conservation of the mechanisms involved. The third stage is the actual cell fusion itself. In this stage, the presence of calcium ions is critical. In mice, fusion is aided by a set of metalloproteinases called meltrins and a variety of other proteins still under investigation. Fusion involves recruitment of actin to the plasma membrane, followed by close apposition and creation of a pore that subsequently rapidly widens.

During embryogenesis, the dermomyotome and/or myotome in the somites contain the myogenic progenitor cells that will evolve into the prospective skeletal muscle. The determination of dermomyotome and myotome is regulated by a gene regulatory network that includes a member of the T-box family, tbx6, ripply1, and mesp-ba. Skeletal myogenesis depends on the strict regulation of various gene subsets in order to differentiate the myogenic progenitors into myofibers. Basic helix-loop-helix (bHLH) transcription factors, MyoD, Myf5, myogenin, and MRF4 are critical to its formation. MyoD and Myf5 enable the differentiation of myogenic progenitors into myoblasts, followed by myogenin, which differentiates the myoblast into myotubes. MRF4 is important for blocking the transcription of muscle-specific promoters, enabling skeletal muscle progenitors to grow and proliferate before differentiating.

### Myogenic Progenitor Cells

Skeletal muscles of adult mammalian species exhibit a capacity to adapt to physiological demands such as growth, training, and injury. The processes by which these adaptations occur are attributed to a small population of mononuclear cells that is resident in adult skeletal muscle and has been referred to as satellite cells. Skeletal muscle fibers are terminally differentiated and the nuclei in these multinucleated cells are incapable of DNA synthesis or mitotic division. Increases in muscle fiber numbers or in numbers of muscle fiber nuclei are due to proliferation and subsequent differentiation of muscle precursor cells known as “myoblasts.” In adults, myoblasts remain as mitotically quiescent reserve precursor populations, which can, upon muscle injury, re-enter the cell cycle, undergo several rounds of proliferation, and subsequently differentiate and permanently exit from the cell cycle. Upon differentiation, differentiated myoblasts acquire the ability to fuse with one another or with preexisting muscle fibers, and also commence expression of a set of muscle-specific myofibrillary and contractile proteins. Quiescent myogenic progenitor cells are physically distinct from the adult myofibers as they reside in indentations between the sarcolemma and the basal lamina. In the case of muscle injury, some of these cells will remain as progenitor cells whereas others will differentiate into new muscle fibers. In response to stimuli such as myotrauma, myogenic progenitor cells become activated, proliferate, and express myogenic markers. Ultimately, these cells fuse to existing muscle fibers or fuse together to form new myofibers during regeneration of damaged skeletal muscle.

### Methods of Producing a Population of Myogenic Progenitor Cells (MPC)

Provided herein are methods of producing a population of myogenic progenitor cells (MPCs). In some embodiments, the method comprises culturing a pluripotent stem cell (PSC) population under suitable conditions to produce a MPC population. In some embodiments, the PSC can be prepared (e.g., dedifferentiated) from cells isolated from a subject.

Pluripotent Stem Cells

The pluripotent stem cells can be induced pluripotent stem (iPS) cells or embryonic stem (ES) cells, without limitation. In certain embodiments, the pluripotent cell is an embryonic stem (ES) cell. In one embodiment, the pluripotent cell is a non-human ES cell. In one embodiment, the pluripotent cell is an induced pluripotent stem (iPS) cell. In one embodiment, the induced pluripotent (iPS) cell is derived from a fibroblast. In one embodiment, the induced pluripotent (iPS) cell is derived from a human fibroblast. In one embodiment, the pluripotent cell is a hematopoietic stem cell (HSC). In one embodiment, the pluripotent cell is a neuronal stem cell (NSC). In one embodiment, the pluripotent cell is an epiblast stem cell. In one embodiment, the pluripotent cell is a developmentally restricted progenitor cell. In one embodiment, the pluripotent cell is a rodent pluripotent cell. In one embodiment, the rodent pluripotent cell is a rat pluripotent cell. In one embodiment, the rat pluripotent cell is a rat ES cell. In one embodiment, the rodent pluripotent cell is a mouse pluripotent cell. In one embodiment, the pluripotent cell is a mouse embryonic stem (ES) cell.

In certain embodiments, hPSCs are plated as single cells for cell culturing by adhesion culture without the use of feeder cells. For the culture, a culture vessel is used such as a dish, a flask, a microplate, or a cell culture sheet such as Geltrex (Gibco), OptiCell (Nalge Nunc International). The culture vessel is surface-treated for improving adhesiveness to cells (hydrophilicity) or coated with a substrate for cell adhesion such as collagen, gelatin, poly-L-lysine, poly-D-lysine, laminin, fibronectin, Matrigel ((e.g., BD Matrigel (Becton Dickinson)) or vitronectin. The culture vessel can be coated with type I collagen, Matrigel, fibronectin, vitronectin or poly-D-lysine. Culturing media includes use of mouse embryonic fibroblast-conditioned media.

In certain embodiments, the cells are cultured in media comprising a growth factor, e.g. fibroblast growth factor 2 (FGF-2) and a ROCK inhibitor e.g., Y-27632. The term “ROCK inhibitor” means a substance inhibiting Rho kinase (ROCK: Rho-associated, coiled-coil containing protein kinase) and may be substance inhibiting any of ROCK I and ROCK II. The ROCK inhibitor is not particularly limited as long as the ROCK inhibitor has the function described above. Examples of the ROCK inhibitor that can be used include: N-(4-pyridinyl)-40-[(R)-1-aminoethyl]cyclohexane-1α-carboxamide (Y-27632), fasudil (HA1077), (2S)-2-methyl-1-[(4-methyl-5-isoquinolinyl]sulfonyl]hexahydro-1-H-1,4-diazepine (H-1152), 40-[(1R)-1-aminoethyl]-N-(4-pyridyl)benzenecarboxamide (Wf-536), N-(1H-pyrrolo[2,3-b]pyridin-4-yl)-4PER(R)-1-aminoethyl]cyclohexane-carbox-amide (Y-30141), N-(3-{[2-(4-amino-1,2,5-oxadiazol-3-yl)-1-ethyl-1H-imidazo[4,5-c]pyridin-6-yl]oxy}phenyl)-4-{[2-(4-morpholinyl)ethyl]-oxy}benzamide (GSK269962A) and N-(6-fluoro-1H-indazol-5-yl)-6-methyl-2-oxo-4-[4-(trifluoromethyl)phenyl]-3,4-dihydro-1H-pyridine-5-carboxamide (GSK429286A); antibodies (including functional fragments), antisense nucleic acids, and siRNA against ROCK; ROCK antagonists and dominant negative forms; and other ROCK inhibitors known in the art.

Differentiation with GSK-3β Inhibitor

Pluripotent stem cells can be induced to differentiate into myogenic progenitor cells (MPCs). In an example procedure, a selective inhibitor of glycogen synthase kinase 3 (GSK-3) e.g. CHIR99021. GSK3β (glycogen synthase kinase 3) is a serine/threonine protein kinase which participates in many signal pathways involved in the production of glycogen, apoptosis, the maintenance of stem cells, etc. GSK3 includes two isoforms, a and R. In certain embodiments, the GSK3β inhibitor is a GSK3β inhibitor. Use of a GSK3β inhibitor is not particularly limited as long as the GSK3β inhibitor has GSK3β inhibitory activity. The GSK3β inhibitor may be a substance having GSK3a inhibitory activity in addition to the GSK3β inhibitory activity.

Examples of the GSK3β inhibitor include CHIR98014(2-[[2-[(5-nitro-6-aminopyridin-2-yl)amino]ethyl]amino]-4-(2,4-dichloroph-enyl)-5-(1H-imidazol-1-yl)pyrimidine), CHIR99021(6-[[2-[[4-(2,4-dichlorophenyl)-5-(4-methyl-1H-imidazol-2-yl)-2-pyrimidin-yl]amino]ethyl]amino]nicotinonitrile), Kenpaullone, AR-A0144-18, TDZD-8(4-benzyl-2-methyl-1,2,4-thiadiazolidine-3,5-dione), SB216763(3-(2,4-dichlorophenyl)-4-(1-methyl-1H-indol-3-yl)-1H-pyrrole-2,5-dione), BIO (6-bromoindirubin-3-oxime), TWS-119(3-[6-(3-aminophenyl)-7H-pyrrolo[2,3-d]pyrimidin-4-yloxy]phenol) and SB415286 (3-[(3-chloro-4-hydroxyphenyl)amino]-4-(2-nitrophenyl)-1H-pyrrol-e-2,5-dione). Also, an antisense oligonucleotide, siRNA, or the like against GSK3β mRNA can be used as the GSK3β inhibitor and is commercially available or can be synthesized according to a method known in the art.

In certain embodiments, the GSK3β inhibitor comprises CHIR99021, SB216763, SB415286, BIO, or a salt thereof.

In certain embodiments, the cells are cultured in a culture medium comprising a GSK3β inhibitor for at least 6 hours, or for at least 12 hours, or for at least 18 hours, or for at least 24 hours, or for at least 48 hours, or for at least 72 hours or for at least 96 hours. In certain embodiments, the cells are cultured in a culture medium comprising a GSK3β inhibitor for 1 to 8 days, 2 to 7 days, 3 to 6 days, 3 to 5 days, 3 to 4 days, or 4 to 5 day, without limitation.

The concentration of the GSK3β inhibitor (e.g., CHIR99021), in some embodiments, may be at least 0.2 NM, 0.5 μM, 1 μM, 1.5 μM, 2 μM, 2.5 μM, or 3 μM. The concentration of the GSK3β inhibitor (e.g., CHIR99021), in some embodiments, may not be higher than 3 μM, 3.5 μM, 4 μM, 4.5 μM, 5 μM, 6 μM, 7 μM, 8 μM, 9 μM, 10 μM, 15 μM, 20 μM, 30 μM or 40 μM. The concentration of the GSK3β inhibitor (e.g., CHIR99021), in some embodiments, may be 0.2-10 μM, 0.5-8 μM, 1-7 μM, 2-5 μM, or 2-4 μM, without limitation.

Treatment with Notch Signaling Inhibitor

Following culturing of the cells with a GSK3β inhibitor, in some examples, the cells are then cultured with a γ-secretase inhibitor and/or Notch signaling inhibitor. An example of a γ-secretase inhibitor is N—[N-(3,5-Difluorophenacetyl)-_{L}-alanyl]-S-phenylglycine t-butyl ester (DAPT). DAPT is a potent and specific inhibitor of γ-secretase that blocks Notch signaling, a multimeric membrane protein complex that catalyzes proteolytic cleavage of amyloid precursor protein (APP) resulting in the accumulation of amyloid-O (AD) peptides which is associated with early on-set of familial Alzheimer's disease (AD). It directly binds to the C-terminal fragment of the catalytic center of γ-secretase, presenilin (PS), especially within the transmembrane domain 7 or more C-terminal region, resulting in the synthesis of a photoactivable DAPT derivative. DAPT indirectly inhibits Notch, which is a substrate for γ-secretase.

A Notch signaling inhibitor is an agent, e.g., a chemical compound or an antibody, that inhibits the Notch signaling pathway. Inhibitors to the γ-secretase, for instance, can inhibit the Notch signaling pathway. Such a γ-secretase inhibitor is for example peptidic in nature or non-peptidic or semi-peptidic and is preferably a small molecule. Examples include DAPT (N—[N-(3,5-difluorophenylacetyl)-L-alanyl]-S-phenylglycine t-butyl ester). Also compounds from the chemical classes AS (arylsulfonamide), DBZ (dibenzazepine (DBZ), BZ (benzodiazepine), LY-411,575 and many others, have been tested for their γ-secretase inhibiting activity. The γ-secretase inhibitors have been divided in solfonamides/sulfones and benzodiazepines/benzolactams. Several of these γ-secretase inhibitors have already been in clinical phase I and UI trials.

In certain embodiments, the cells are cultured in media comprising at least a Notch signaling inhibitor (e.g., DAPT) for at least about 1 day, or for at least about two days, or for at least about three days, or for at least about four days, of for at least about five days, or for at least about 6 days, or for at least about seven days, or for at least about eight days. In certain embodiments, the cells are cultured in media comprising at least a Notch signaling inhibitor (e.g., DAPT) for 1 to 20 days, 2 to 15 days, 3 to 12 days, 4 to 11 days, 5 to 10 days, 6 to 9 days, 7 to 9 days, 7-8 days, or 8-10 days, without limitation.

Sorting and Expansion

Following the treatment with the GSK3β inhibitor and the Notch signaling inhibitor, in some embodiments, the cells are allowed to further differentiate for a number of days (after the agents are removed from the media). In some embodiments, the additional differentiation may last at least about 2 days, 4 days, 6 days, 8 days, 10 day, 12 days, 13 days, 14 days, 15 days, 16 days, 17 days, 18 days, 20 days, 25 days or 30 days.

The differentiated PAX7+ cells, in some embodiments, can be sorted (enriched) and expanded. In some embodiments, the cells are sorted, followed by expansion. In some embodiments, the cells are expanded, followed by sorting.

Sorting of the differentiated cells can be carried out with agents recognizing the PAX7+ MPC. Examples of such agents include antibodies specific to cell surface markers. The PAX7+ myogenic progenitor cells can be enriched from other cells, such as the pluripotent cells and cells still undergoing differentiation. For example, the antibodies can be used in FACS or magnetic beads to sort, isolate and purify progenitor cells. In some embodiments, the antibodies used to specific to the panel of markers of the target myogenic progenitor cells, such as NCAM⁺, HNK1⁻, PAX7+, MyoD+, CD54+, integrin α9β1+ and SDC2+ (Syndecan2).

Expansion of the PAX7+ myogenic progenitor cells can be done in a medium that is supplemented with FGF2 (also known as basic fibroblast growth factor (bFGF) and FGF-β) or FGF8, or the combination thereof, optionally with FBS.

### MPC Population Capable of In Vivo Regeneration and Engraftment

The enriched and expanded PAX7+ MPC, as shown in the experimental examples, have excellent repopulation and engraftment capabilities. The instant inventors believe that this was the first time that myogenic progenitor cells derived from pluripotent stem cells in vitro can reside in muscle stem cell niche upon intra-muscular transplantation, become quiescent and change their molecular expression profile that is similar to human muscle stem cells.

Also as shown in the examples, the regeneration and engraftment capability of the PAX7+ MPCs requires a minimum cell concentration. As shown, when the concentration of the PAX7+ MPCs was under 15%, no regeneration and engraftment was observed.

In accordance with one embodiment of the present disclosure, therefore, provided is a population of cells (e.g., mammalian cells, or more particularly human cells) wherein at least 30% of the cells are PAX7+ myogenic progenitor cells (MPCs) derived from pluripotent stem cells in vitro or ex vivo. In some embodiments, the cell population includes at least 35%, 40%, 45%, 50%, 55%, 60%, 65%, 70%, 75%, 80%, 85%, 90% or 95% PAX7+ MPCs.

In some embodiments, a cell population includes at least 100, 1000, 10,000, 100,000, 1×10⁶, 1×10⁷, 1×10⁸, or 1×10⁹ cells. In some embodiments, a substantial portion of the cells of the population have been cultured along with the PAX7+ MPCs during the differentiation. For instance, at least 50%, 60%, 70%, 80%, 90%, 95%, 98%, 99%, 99.5%, or 99.9% of the cells of the population have been cultured along with the PAX7+ MPCs during the differentiation.

The PAX7+ MPCs obtained by the present technology are believed to be different from natural myogenic progenitor cells. In addition to PAX7+, NCAM⁺, HNK1⁻, PAX7+, MyoD+, CD54+, integrin α9β1+ and SDC2+ They can be characterized with one or more of the following cell surface markers: CHRNA1+, NTSR1+, FZD1+, FZD5−, GPR37− and GPR27−. In some embodiments, the derived PAX7+ MPCs are characterized by at least two, three, four, or five of CHRNA1+, NTSR1+, FZD1+, FZD5−, GPR37− and GPR27−.

In some embodiments, the at least two are CHRNA1+ and NTSR1+, CHRNA1+ and FZD1+, CHRNA1+ and FZD5−, CHRNA1+ and GPR37−, CHRNA1+ and GPR27−, NTSR1+ and CHRNA1+, NTSR1+ and FZD1+, NTSR1+ and FZD5−, NTSR1+ and GPR37−, NTSR1+ and GPR27−, FZD1+ and CHRNA1+, FZD1+ and NTSR1+, FZD1+ and FZD5−, FZD1+ and GPR37−, FZD1+ and GPR27−, FZD5− and CHRNA1+, FZD5− and NTSR1+, FZD5− and FZD1+, FZD5− and GPR37−, FZD5− and GPR27−, GPR37− and CHRNA1+, GPR37− and NTSR1+, GPR37− and FZD1+, GPR37− and FZD5−, GPR37− and GPR27−, GPR27− and CHRNA1+, GPR27− and NTSR1+, GPR27− and FZD1+, GPR27− and FZD5−, or GPR27− and GPR37−.

In some embodiments, the derived PAX7+ MPCs are characterized by at least three of CHRNA1+, NTSR1+, FZD1+, FZD5−, GPR37− and GPR27−. In some embodiments, the derived PAX7+ MPCs are characterized by at least four of CHRNA1+, NTSR1+, FZD1+, FZD5−, GPR37− and GPR27−. In some embodiments, the derived PAX7+ MPCs are characterized by at least five of CHRNA1+, NTSR1+, FZD1+, FZD5−, GPR37− and GPR27−. In some embodiments, the derived PAX7+ MPCs are characterized by all of CHRNA1+, NTSR1+, FZD1+, FZD5−, GPR37− and GPR27−.

Whether a cell surface protein is positive (+) or negative (−) can be assessed by agents recognizing the marker, such as an antibody. It is readily appreciated by the skilled artisan, however, such positive and negative may not be absolute. In some embodiments, a marker being positive is to have a higher expression on the cell than on a reference cell. In some embodiments, a marker being negative is to have a lower expression on the cell than on a reference cell. The reference cell, for instance, is a cell likewise differentiated from a pluripotent stem cell but cannot regenerate a muscle tissue in vivo.

The methods for producing an MPC population is effective to increase PAX7 expression in cells of the MPC population. For example, the expression may be increased by at least 0, 20, 30, 40, 50, 60, 70, 80, 90, 100, 125, 150, 175, or 200% compared to a reference. In embodiments, PAX7 expression in the myogenic progenitor cell population increases by at least 100%. In embodiments, the level of PAX7 expression in the myogenic progenitor cell population increases by at least 125%. In embodiments, the level of PAX7 expression in the myogenic progenitor cell population increases by at least 150%. In embodiments, the level of PAX7 expression in the myogenic progenitor cell population increases by at least 50%, 100%, 200%, 300%, 400%, 500%, 600%, 700%, 800%, 900%, 1000%, 1100%, or 1200%. In embodiments, the level of PAX7 expression in the myogenic progenitor cell population increases by about 50% to about 1200%.

An amino acid sequence for human PAX7 is publicly available in the NCBI GenBank database under accession number NP_002575.1 (SEQ ID NO: 1) and is as follows:

| | |
|---|---|
| 1 | MAALPGTVPR MMRPAPGQNY PRTGFPLEVS TPLGQGRVNQ |
| | LGGVFINGRP LPNHIRHKIV |
| 61 | EMAHHGIRPC VISRQLRVSH GCVSKILCRY QETGSIRPGA |
| | IGGSKPRQVA TPDVEKKIEE |
| 121 | YKRENPGMFS WEIRDRLLKD GHCDRSTVPS GLVSSISRVL |
| | RIKFGKKEEE DEADKKEDDG |
| 181 | EKKAKHSIDG ILGDKGNRLD EGSDVESEPD LPLKRKQRRS |
| | RTTFTAEQLE ELEKAFERTH |
| 241 | YPDIYTREEL AQRTKLTEAR VQVWFSNRRA RWRKQAGANQ |
| | LAAFNHLLPG GFPPTGMPTL |
| 301 | PPYQLPDSTY PTTTISQDGG STVHRPQPLP PSTMHQGGLA |
| | AAAAAADTSS AYGARHSFSS |
| 361 | YSDSFMNPAA PSNHMNPVSN GLSPQVMSIL GNPSAVPPQP |
| | QADFSISPLH GGLDSATSIS |
| 421 | ASCSQRADSI KPGDSLPTSQ AYCPPTYSTT GYSVDPVAGY |
| | QYGQYGQSEC LVPWASPVPI |
| 481 | PSPTPRASCL FMESYKVVSG WGMSISQMEK LKSSQMEQFT |

A nucleotide sequence that encodes human PAX7 is publicly available in the GenBank database under accession number NM_002584.2 (SEQ ID NO: 2) and is as follows (start and stop codon are bolded and underlined):

| | |
|---|---|
| 1 | gaaagctggt gtggagggag aagcgagtgt ggtccggaga aagaaggcgt ggagaagagg |
| 61 | gagggagcga gagcgagaga ataaatatat aaataaatac gagaacgaaa tccactccgc |
| 121 | agtctccggg ctcggaaact ttggccccga gcgccagagc gccagagcgc gagagcgcgg |
| 181 | cgctcgccac tctgaggctg gcggcctcga ttccggccgc gttcccccgg cccccctccg |
| 241 | ccgcggggcc tggtctccgg gttctgccag gcgcatcagc ccgcacaact tctggccgag |
| 301 | gccagccggc agaggcggac ttggggttgg agtgtttgtt tgtttgaact tcctcgtcgt |
| 361 | cgccaccttc cctcccccca acctccaccc cacctcaccc ccctccccag cttctggacg |
| 421 | cgtttgactg cagccagggg tggggggtgg gggtagggag tgtgtgtgga ggggagggag |
| 481 | aagaggttaa aaaaaagaag acgaagaaga cggaaagaaa gagatcgcag caggggtgaa |
| 541 | gggagcggac gggaagcgat ttttgccgac tttggattcg tccccggcgt gcgcaagaat |
| 601 | ggcggccctt cccggcacgg taccgagaat gatgcggccg gctccggggc agaactaccc |
| 661 | ccgcacggga ttccctttgg aagtgtccac cccgcttggc caaggccggg tcaatcagct |
| 721 | gggaggggtc ttcatcaatg ggcgacccct gcctaaccac atccgccaca agatagtgga |
| 781 | gatggcccac catggcatcc ggccctgtgt catctcccga cagctgcgtg tctcccacgg |
| 841 | ctgcgtctcc aagattcttt gccgctacca ggagaccggg tccatccggc ctggggccat |
| 901 | cggcggcagc aagcccagac aggtggcgac tccggatgta gagaaaaaga ttgaggagta |
| 961 | caagagggaa aacccaggca tgttcagctg ggagatccgg gacaggctgc tgaaggatgg |
| 1021 | gcactgtgac cgaagcactg tgccctcagg tttagtgagt tcgattagcc gcgtgctcag |
| 1081 | aatcaagttc gggaagaaag aggaggagga tgaagcggac aagaaggagg acgacggcga |
| 1141 | aaagaaggcc aaacacagca tcgacggcat cctgggcgac aaagggaacc ggctggacga |
| 1201 | gggctcggat gtggagtcgg aacctgacct cccactgaag cgcaagcagc gacgcagtcg |
| 1261 | gaccacattc acggccgagc agctggagga gctggagaag gcctttgaga ggacccacta |
| 1321 | cccagacata tacacccgcg aggagctggc gcagaggacc aagctgacag aggcgcgtgt |
| 1381 | gcaggtctgg ttcagtaacc gccgcgcccg ttggcgtaag caggcaggag ccaaccagct |
| 1441 | ggcggcgttc aaccaccttc tgccaggagg cttcccaccc accggcatgc ccacgctgcc |
| 1501 | cccctaccag ctgccggact ccacctaccc caccaccacc atctcccaag atgggggcag |
| 1561 | cactgtgcac cggcctcagc ccctgccacc gtccaccatg caccagggcg ggctggctgc |
| 1621 | agcggctgca gccgccgaca ccagctctgc ctacggagcc cgccacagct tctccagcta |
| 1681 | ctctgacagc ttcatgaatc cggcggcgcc ctccaaccac atgaacccgg tcagcaacgg |
| 1741 | cctgtctcct caggtgatga gcatcttggg caaccccagt gcggtgcccc cgcagccaca |
| 1801 | ggctgacttc tccatctccc cgctgcatgg cggcctggac tcggccacct ccatctcagc |
| 1861 | cagctgcagc cagcgggccg actccatcaa gccaggagac agcctgccca cctcccaggc |
| 1921 | ctactgccca cccacctaca gcaccaccgg ctacagcgtg gaccccgtgg ccggctatca |
| 1981 | gtacggccag tacggccaga gtgagtgcct ggtgccctgg gcgtcccccg tccccattcc |
| 2041 | ttctcccacc cccagggcct cctgcttgtt tatggagagc tacaaggtgg tgtcagggtg |
| 2101 | gggaatgtcc atttcacaga tggaaaaatt gaagtccagc cagatggaac agttcaccta |
| 2161 | aaatgacact gagttgggca aaacccagga catctcctgg ctaagcctct gcttccgtac |
| 2221 | tatggctcca acagaaataa aatacacaac acaaatatca aaaaaaaaaa a |

50 An amino acid sequence for mouse PAX7 is publicly available in the NCBI GenBank database under accession number NP_035169.1 (SEQ ID NO: 3) and is as follows:

| | |
|---|---|
| 1 | MAALPGAVPR MMRPGPGQNY PRTGFPLEVS TPLGQGRVNQ |
| | LGGVFINGRP LPNHIRHKIV |
| 61 | EMAHHGIRPC VISRQLRVSH GCVSKILCRY QETGSIRPGA |
| | IGGSKPRQVA TPDVEKKIEE |
| 121 | YKRENPGMFS WEIRDRLLKD GHCDRSTVPS VSSISRVLRI |
| | KFGKKEDDEE GDKKEEDGEK |
| 181 | KAKHSIDGIL GDKGNRLDEG SDVESEPDLP LKRKQRRSRT |
| | TFTAEQLEEL EKAFERTHYP |
| 241 | DIYTREELAQ RTKLTEARVQ VWFSNRRARW RKQAGANQLA |
| | AFNHLLPGGF PPTGMPTLPP |
| 301 | YQLPDSTYPT TTISQDGGST VHRPQPLPPS TMHQGGLAAA |
| | AAAADTSSAY GARHSFSSYS |
| 361 | DSFMNPGAPS NHMNPVSNGL SPQVMSILSN PSAVPPQPQA |
| | DFSISPLHGG LDSASSISAS |
| 421 | CSQRADSIKP GDSLPTSQSY CPPTYSTTGY SVDPVAGYQY |
| | SQYGQTAVDY LAKNVSLSTQ |
| 481 | RRMKLGEHSA VLGLLPVETG QAY |

A nucleotide sequence that encodes mouse PAX7 is publicly available in the GenBank database under accession number NM_011039.2 (SEQ ID NO: 4) and is as follows (start and stop codon are bolded and underlined):

| | |
|---|---|
| 1 | agagacgcca agaggtttat ccagccgact ctggattcgt ctccagcgtg cgcaggaatg |
| 161 | gcggcgctgc ccggcgcggt ccccaggatg atgagacccg gcccggggca gaactacccg |
| 121 | cgcaccggct tccccctgga agtgtccacc cctcttggcc aaggccgggt caatcagctt |
| 181 | ggtggggtct tcatcaacgg tcgacccctg ccgaaccaca tccgtcacaa gatagtggaa |
| 241 | atggcccacc atggcatccg gccctgcgtc atctcccgtc agctccgtgt ttcccatggt |
| 301 | tgtgtctcca agattctgtg ccgatatcag gagactgggt ccatccggcc cggggctatc |
| 361 | ggaggcagca agcccagaca ggtggcgact ccggatgtgg agaaaaagat tgaggagtat |
| 421 | aagagagaga accccgggat gttcagctgg gaaatccggg accggctgct gaaggacggt |
| 481 | cactgcgacc gaagcacggt gccctcagtg agttcgatta gccgagtgct cagaatcaag |
| 541 | ttcgggaaga aagaggacga cgaggaagga gacaagaaag aagaagatgg cgagaagaaa |
| 601 | gccaaacaca gcatcgatgg catcctgggc gacaaaggga accgtctgga tgagggctca |
| 661 | gatgtggaat cagaacccga cctccccctg aagcgcaagc agcgccgcag tcggaccacg |
| 721 | ttcacagccg agcagctgga ggagctggag aaggccttcg agaggaccca ctacccggac |
| 781 | atctacaccc gggaggagct ggcacagagg accaagctca cggaggcacg cgtccaggtc |
| 841 | tggttcagta accggcgtgc ccgctggcgt aagcaggcag gagctaacca gctggccgcc |
| 901 | ttcaaccacc ttctgccggg aggtttccca cccaccggca tgcccacgct gccaccctac |
| 961 | cagctgccgg actctaccta ccccaccacc accatctccc aagatggagg cagcacagta |
| 1021 | cacaggcccc agccccttcc gccatcaacc atgcatcagg gtgggctggc tgcggccgct |
| 1081 | gcagcagcgg acaccagctc tgcctacgga gcccgccaca gcttctccag ctactctgac |
| 1141 | agcttcatga accctggggc tccctccaac cacatgaacc ctgtcagcaa tggcctgtct |
| 1201 | cctcaggtca tgagcatcct tagcaacccg agtgccgtgc ctccacagcc ccaggccgac |
| 1261 | ttctccatct ccccgctgca tggaggcctg gactcggctt cctccatctc agccagctgc |
| 1321 | agccaacggg ccgactccat caagccagga gacagcttgc ccacgtccca gtcttactgc |
| 1381 | ccacccacct acagcaccac tggctacagt gtggaccctg tggctggcta ccagtacagc |
| 1441 | cagtatggcc aaactgctgt tgattacctg gccaaaaacg tgagcctgtc cacacagcgc |
| 1501 | cgtatgaagc ttggggaaca ctccgctgtg ctgggacttc ttcctgtgga aacgggacaa |
| 1561 | gcctactagg gtccctgggg caacttgccc catccagtgg cccagccaac ccttcccaag |
| 1621 | ccctgagtct cctcacctca gtccccttat ccccctgggg ttgcaggaga ccaagggaaa |
| 1681 | aaaacccttt cccttcctac aggaaaccct ctggagacgg aaaaccagtg tgccatctac |
| 1741 | ccatgcttag tgacccagag tggccccttg ccttcccctc tttcttcaga ggggttccta |
| 1801 | ggcatcctgc agtgacctcc agctcacatc caccttctct gtatcgtggc ctcggtcctg |
| 1861 | tctcagtgca gagattgagg ctcaatttga accaagcacc tagttatcag aagaaaatgg |
| 1921 | tgccaaagac aaggccctgg agtccttgac ctctgagtcg tgggtgccct ggctatgggt |
| 1981 | gtaggtggag cccatgggtg tcctcagtca cagagctggg agctctctct cgctcgcttg |
| 2041 | gcatcaggac tgcagcctct ttcactggac actgagatga gtccccaggg tgttcccagg |
| 2101 | ggagaaagca ggtaacatcc cagctttacc taggaatcca gaggacttta ggactgtccc |
| 2161 | tatgcaccct gcagggcatc aggagaccag gaagggattc tagcagaggg tagggggcac |
| 2221 | agaggcagag ctgattgccc atgggctatc ccagaatgcc tggtcctgaa tctagcatca |
| 2281 | ggaggtgcag gattcctagg ctgcaatctg acagaggctt gcccactgtg tcaggcctgg |
| 2341 | gcagcccaca gaacctgtca ctctcctcaa ttggtaggag aagaggtctt gaggtgacag |
| 2401 | gaggcagaag gcaggctcag acagtcagag agcaccaagc tttcaagtcc gcacccctgg |
| 2461 | ggttcggcat accatcttgc tggcagctgg aaacctggtt ccctgaaagg gggcctccat |
| 2521 | cctccagaat gtaaggctct tgatgccacc ggatgcagag agccttctcg ggccagacaa |
| 2581 | aattgctgct ccaccccaga gaagatgttc cagccttctt ggcatcttag aggaaggcca |
| 2641 | tgtcgctgtc cttttcagag tagcatattt ttcagtgatg gctgctcagt caggaggctt |
| 2701 | ctgtcgcctt acaaagcaca gtgcgctctg ggcactgttt ctaagccacc ccatcccacc |
| 2761 | cccacccccg ccaccccggg gacagaggaa gatgctaaaa gtcccagcaa agaggacaaa |
| 2821 | gcacctttct taagcactcc agagtcttcc ttgtaccccg ccctctctta gagctgggtc |
| 2881 | ttttgaggga aacggattgc tgagccctcc ccccccccca atcctctcct ctgtggagct |
| 2941 | gtttatcatc ctctatttat caaaatcgca tccatcttta ccctctcctt cgctatagcc |
| 3001 | tacttctgga tcaccctcat ccagtgctgg taccccacag cactaaatcc aggaaccctg |
| 3061 | ggcttgatca cctgttgcca cctgtacaca tgaaaccacc tgctggcccg gcccatgtct |
| 3121 | cctgccctca gccagcaaga cattcctaga gagaggaact atgggcttaa aagccccaac |
| 3181 | tgacttcctt ttgcctgggg acctgaaccg acaagacacc agggacactt gtctacatga |
| 3241 | acatgtgacc aatgtacacc gatttctcat ctctagacct attatctgaa gcctgtcccg |
| 3301 | ggccatgact agaatggctt gtatctgtgg tttagagaag tctaataata actgagggca |
| 3361 | aactgactct ctggtagcat ggagcaccag gcggatggag ctcaccagct ctgtccaggt |
| 3421 | ttcaaaggag gagactgttg ggctcttcaa ggtctggaca agaggaaagc cacattgccc |
| 3481 | ccttgggaac ccaggttctc cttttgaact tctcacagct gcaagcaccc ctttcaaaga |
| 3541 | ccaaatgcat cctcctccac attccttgct ccctggaggc ctggctctgg atacacctga |
| 3601 | gtcttcgttc acctactaca ctttaggagc aggaacttca agcaggtgac atccacaggg |
| 3661 | cccagtccca gccaagggag caacattcca acgcttggac caatcataat gatctgcccg |
| 3721 | tgagggtaac cgcaactaga gacctgcttg ggagaaaaca aaatgacttc tcattccatg |
| 3781 | ccatgcctct gaatgctccc ccaagctgcc atcttggtat aaaatgggac ttgtgttgtg |
| 3841 | gggaacccct tgaccccaac aggttttccc aactgtctca tgcttttgtg aatctgtctg |
| 3901 | ctttgatctg taaaactcag ccttgtttgg gcagcttgta atttcaacag tgaggcgaca |
| 3961 | tcgattagat gagaggcacc aggcctctcc gccgccgtcc ctctgtggcc gtccctctgg |
| 4021 | ggttgagcag aacctagaag aaggccgatt tccagtggcc agactggacc agaaacagcc |
| 4081 | cccaccccaa tccctgtaaa tagagtcaat agcaaaataa gaggggcgcc ctccatgtca |
| 4141 | cctcaagtag ctactggttc ttctgtggag gcccctctga actcattgtc tggtagttga |
| 4201 | aaatgtgatg ttgtgctgtt tgtttataga acattggctt tttatatata aatctatata |
| 4261 | cttaaaaaca aaaac |

The methods for producing an MPC population is effective to increase MyoD expression in cells of the MPC population. For example, the expression may be increased by at least 0, 20, 30, 40, 50, 60, 70, 80, 90, 100, 125, 150, 175, or 200% compared to a reference. In embodiments, MyoD expression in the myogenic progenitor cell population increases by at least 100%. In embodiments, the level of MyoD expression in the myogenic progenitor cell population increases by at least 125%. In embodiments, the level of MyoD expression in the myogenic progenitor cell population increases by at least 150%. In embodiments, the level of MyoD expression in the myogenic progenitor cell population increases by at least 50%, 100%, 200%, 300%, 400%, 500%, 600%, 700%, 800%, 900%, 1000%, 1100%, or 1200%. In embodiments, the level of MyoD expression in the myogenic progenitor cell population increases by about 50% to about 1200%.

An amino acid sequence for human MyoD is publically available in the NCBI GenBank database under accession number NP_002469.2 (SEQ ID NO: 5) and is as follows:

| | |
|---|---|
| 1 | MELLSPPLRD VDLTAPDGSL CSFATTDDFY DDPCFDSPDL |
| | RFFEDLDPRL MHVGALLKPE |
| 61 | EHSHFPAAVH PAPGAREDEH VRAPSGHHQA GRCLLWACKA |
| | CKRKTTNADR RKAATMRERR |
| 121 | RLSKVNEAFE TLKRCTSSNP NQRLPKVEIL RNAIRYIEGL |
| | QALLRDQDAA PPGAAAAFYA |
| 181 | PGPLPPGRGG EHYSGDSDAS SPRSNCSDGM MDYSGPPSGA |
| | RRRNCYEGAY YNEAPSEPRP |
| 241 | GKSAAVSSLD CLSSIVERIS TESPAAPALL LADVPSESPP |
| | RRQEAAAPSE GESSGDPTQS |
| 301 | PDAAPQCPAG ANPNPIYQVL |

A nucleotide sequence that encodes human MyoD is publically available in the GenBank database under accession number NM_002478.5 (SEQ ID NO: 6) and is as follows (start and stop codon are bolded and underlined):

| | |
|---|---|
| 1 | aggggtgagg aagccctggg gcgctgccgc cgctttcctt aaccacaaat caggccggac |
| 61 | aggagaggga ggggtggggg acagtgggtg ggcattcaga ctgccagcac tttgctatct |
| 121 | acagccgggg ctcccgagcg gcagaaagtt ccggccactc tctgccgctt gggttgggcg |
| 181 | aagccaggac cgtgccgcgc caccgccagg atatggagct actgtcgcca ccgctccgcg |
| 241 | acgtagacct gacggccccc gacggctctc tctgctcctt tgccacaacg gacgacttct |
| 301 | atgacgaccc gtgtttcgac tccccggacc tgcgcttctt cgaagacctg gacccgcgcc |
| 361 | tgatgcacgt gggcgcgctc ctgaaacccg aagagcactc gcacttcccc gcggcggtgc |
| 421 | acccggcccc gggcgcacgt gaggacgagc atgtgcgcgc gcccagcggg caccaccagg |
| 481 | cgggccgctg cctactgtgg gcctgcaagg cgtgcaagcg caagaccacc aacgccgacc |
| 541 | gccgcaaggc cgccaccatg cgcgagcggc gccgcctgag caaagtaaat gaggcctttg |
| 601 | agacactcaa gcgctgcacg tcgagcaatc caaaccagcg gttgcccaag gtggagatcc |
| 661 | tgcgcaacgc catccgctat atcgagggcc tgcaggctct gctgcgcgac caggacgccg |
| 721 | cgccccctgg cgccgcagcc gccttctatg cgccgggccc gctgcccccg ggccgcggcg |
| 781 | gcgagcacta cagcggcgac tccgacgcgt ccagcccgcg ctccaactgc tccgacggca |
| 841 | tgatggacta cagcggcccc ccgagcggcg cccggcggcg gaactgctac gaaggcgcct |
| 901 | actacaacga ggcgcccagc gaacccaggc ccgggaagag tgcggcggtg tcgagcctag |
| 961 | actgcctgtc cagcatcgtg gagcgcatct ccaccgagag ccctgcggcg cccgccctcc |
| 1021 | tgctggcgga cgtgccttct gagtcgcctc cgcgcaggca agaggctgcc gcccccagcg |
| 1081 | agggagagag cagcggcgac cccacccagt caccggacgc cgccccgcag tgccctgcgg |
| 1141 | gtgcgaaccc caacccgata taccaggtgc tctgagggga tggtggccgc ccacccgccc |
| 1201 | gagggatggt gcccctaggg tccctcgcgc ccaaaagatt gaacttaaat gcccccctcc |
| 1261 | caacagcgct ttaaaagcga cctctcttga ggtaggagag gcgggagaac tgaagtttcc |
| 1321 | gcccccgccc cacagggcaa ggacacagcg cggttttttc cacgcagcac ccttctcgga |
| 1381 | gacccattgc gatggccgct ccgtgttcct cggtgggcca gagctgaacc ttgaggggct |
| 1441 | aggttcagct ttctcgcgcc ctcccccatg ggggtgagac cctcgcagac ctaagccctg |
| 1501 | ccccgggatg caccggttat ttgggggggc gtgagaccca gtgcactccg gtcccaaatg |
| 1561 | tagcaggtgt aaccgtaacc cacccccaac ccgtttcccg gttcaggacc actttttgta |
| 1621 | atacttttgt aatctattcc tgtaaataag agttgctttg ccagagcagg agcccctggg |
| 1681 | gctgtattta tctctgaggc atggtgtgtg gtgctacagg gaatttgtac gtttataccg |
| 1741 | caggcgggcg agccgcgggc gctcgctcag gtgatcaaaa taaaggcgct aatttatacc |
| 1801 | gcc |

An amino acid sequence for mouse MyoD is publically available in the NCBI GenBank database under accession number NP_034996.2 (SEQ ID NO: 7) and is as follows:

| | |
|---|---|
| 1 | MELLSPPLRD IDLTGPDGSL CSFETADDFY DDPCFDSPDL |
| | RFFEDLDPRL VHMGALLKPE |
| 61 | EHAHFPTAVH PGPGAREDEH VRAPSGHHQA GRCLLWACKA |
| | CKRKTTNADR RKAATMRERR |
| 121 | RLSKVNEAFE TLKRCTSSNP NQRLPKVEIL RNAIRYIEGL |
| | QALLRDQDAA PPGAAAFYAP |
| 181 | GPLPPGRGSE HYSGDSDASS PRSNCSDGMM DYSGPPSGPR |
| | RQNGYDTAYY SEAARESRPG |
| 241 | KSAAVSSLDC LSSIVERIST DSPAAPALLL ADAPPESPPG |
| | PPEGASLSDT EQGTQTPSPD |
| 301 | AAPQCPAGSN PNAIYQVL |

A nucleotide sequence that encodes mouse MyoD is publically available in the GenBank database under accession number NM_010866.2 (SEQ ID NO: 8) and is as follows (start and stop codon are bolded and underlined):

| | |
|---|---|
| 1 | aggggccagg acgccccagg acacgactgc tttcttcacc actcctctga caggacagga |
| 61 | cagggaggag gggtagagga cagccggtgt gcattccaac ccacagaacc tttgtcattg |
| 121 | tactgttggg gttccggagt ggcagaaagt taagacgact ctcacggctt gggttgaggc |
| 181 | tggacccagg aactgggata tggagcttct atcgccgcca ctccgggaca tagacttgac |
| 241 | aggccccgac ggctctctct gctcctttga gacagcagac gacttctatg atgacccgtg |
| 301 | tttcgactca ccagacctgc gcttttttga ggacctggac ccgcgcctgg tgcacatggg |
| 361 | agccctcctg aaaccggagg agcacgcaca cttccctact gcggtgcacc caggcccagg |
| 421 | cgctcgtgag gatgagcatg tgcgcgcgcc cagcgggcac caccaggcgg gtcgctgctt |
| 481 | gctgtgggcc tgcaaggcgt gcaagcgcaa gaccaccaac gctgatcgcc gcaaggccgc |
| 541 | caccatgcgc gagcgccgcc gcctgagcaa agtgaatgag gccttcgaga cgctcaagcg |
| 601 | ctgcacgtcc agcaacccga accagcggct acccaaggtg gagatcctgc gcaacgccat |
| 661 | ccgctacatc gaaggtctgc aggctctgct gcgcgaccag gacgccgcgc cccctggcgc |
| 721 | cgctgccttc tacgcacctg gaccgctgcc cccaggccgt ggcagcgagc actacagtgg |
| 781 | cgactcagat gcatccagcc cgcgctccaa ctgctctgat ggcatgatgg attacagcgg |
| 841 | ccccccaagc ggcccccggc ggcagaatgg ctacgacacc gcctactaca gtgaggcggc |
| 901 | gcgcgagtcc aggccaggga agagtgcggc tgtgtcgagc ctcgactgcc tgtccagcat |
| 961 | agtggagcgc atctccacag acagccccgc tgcgcctgcg ctgcttttgg cagatgcacc |
| 1021 | accagagtcg cctccgggtc cgccagaggg ggcatcccta agcgacacag aacagggaac |
| 1081 | ccagaccccg tctcccgacg ccgcccctca gtgtcctgca ggctcaaacc ccaatgcgat |
| 1141 | ttatcaggtg ctttgagaga tcgactgcag cagcagaggg cgcaccaccg taggcactcc |
| 1201 | tggggatggt gtccctggtt cttcacgccc aaaagatgaa gcttaaatga cactcttccc |
| 1261 | aactgtcctt tcgaagccgt tcttccagag ggaagggaag agcagaagtc tgtcctagat |
| 1321 | ccagccccaa agaaaggaca tagtcctttt tgttgttgtt gttgtagtcc ttcagttgtt |
| 1381 | tgtttgtttt ttcatgcggc tcacagcgaa ggccacttgc actctggctg cacctcactg |
| 1441 | ggccagagct gatccttgag tggccaggcg ctcttccttt cctcatagca caggggtgag |
| 1501 | ccttgcacac ctaagccctg ccctccacat ccttttgttt gtcactttct ggagccctcc |
| 1561 | tggcacccac ttttccccac agcttgcgga ggccactcag gtctcaggtg taacaggtgt |
| 1621 | aaccataccc cactctcccc cttcccgcgg ttcaggacca cttatttttt tatataagac |
| 1681 | ttttgtaatc tattcgtgta aataagagtt gcttggccag agcgggagcc ccttgggcta |
| 1741 | tatttatctc ccaggcatgc tgtgtagtgc aacaaaaact ttgtatgttt attcctcaag |
| 1801 | cgggcgagcc tcgaggctcg ctcgctcagg tgttggaaat aaagacgcta attt |

In other aspects, the methods tor producing an MPC population include that the cell population is cultured and expanded ex vivo for at least 30 days. In other aspects, the cell population is cultured and expanded ex vivo for at least 5 days, at least 10 days, at least 20 days, at least 30 days, at least 40 days, at least 50 days.

In aspects, the cell population is cultured to expand their number. For example, the number may be increased by at least 0, 20, 30, 40, 50, 60, 70, 80, 90, 100, 125, 150, 175, or 200%. In embodiments, the number may increase by at least 50%, 100%, 200%, 300%, 400%, 500%, 600%, 700%, 800%, 900%, 1000%, 1100%, or 1200%. In embodiments, the cell population is cultured and expanded by at least 10%, by at least 20%, by at least 30%, by at least 40%, by at least 50%.

In additional examples, the MPC population is produced from a population of cells obtained from the subject, e.g., skeletal cells. In other examples, the PSC population is an induced pluripotent stem (iPS) cell population.

In further aspects, the cell population is obtained from the subject via biopsy.

Also provided herein is a population of myogenic progenitor cells produced according to the methods described herein.

### Methods for Treating Muscle Diseases or Disorders

Included herein is a method of preventing or treating muscle diseases or disorders in a subject in need thereof. In further embodiments, the method comprises administering to the subject an effective amount of the population of myogenic progenitor cells produced according to the methods described herein. The myogenic progenitor cells may be derived/differentiated from a pluripotent stem cell that is prepared from a cell from a biological sample.

Biological Sample

In certain embodiments, a biological sample is obtained from one or more sources comprising: autologous, allogeneic, haplotype matched, haplotype mismatched, haplo-identical, xenogeneic, cell lines or combinations thereof.

Accordingly, the cells in some embodiments are primary cells, e.g., primary human cells. The samples include tissue, fluid, and other samples taken directly from the subject, as well as samples resulting from one or more processing steps, such as separation, centrifugation, genetic engineering (e.g. transduction with viral vector), washing, and/or incubation. The biological sample can be a sample obtained directly from a biological source or a sample that is processed. Biological samples include, but are not limited to, body fluids, such as blood, plasma, serum, cerebrospinal fluid, synovial fluid, urine and sweat, tissue and organ samples, including processed samples derived therefrom.

Skeletal muscle (SkM) regeneration relies on the activity of myogenic progenitors that reside beneath the basal lamina of myofibers. Alterations in myoblast proliferation, differentiation, and fusion are features shared by many neuromuscular disorders, and can be used to assay cell-based and pharmacological therapies. Human skeletal muscle biopsies, especially those affected by disease, often contain extensive populations of non-myogenic cells such as adipocytes and fibroblasts. Accordingly, in certain embodiments, embodiments, the sample is a muscle tissue or bone tissue.

In some aspects, the sample from which the cells are derived or isolated is blood or a blood-derived sample, or is or is derived from an apheresis or leukapheresis product. Exemplary samples include whole blood, peripheral blood mononuclear cells (PBMCs), leukocytes, bone marrow, thymus, tissue biopsy, tumor, leukemia, lymphoma, lymph node, gut associated lymphoid tissue, mucosa associated lymphoid tissue, spleen, other lymphoid tissues, liver, lung, stomach, intestine, colon, kidney, pancreas, breast, bone, prostate, cervix, testes, ovaries, tonsil, or other organ, and/or cells derived therefrom. Samples include, in the context of cell therapy, e.g., adoptive cell therapy, samples from autologous and allogeneic sources.

In some embodiments, the cells are derived from cell lines. The cells in some embodiments are obtained from a xenogeneic source, for example, from mouse, rat, non-human primate, or pig.

In some embodiments, isolation of the cells includes one or more preparation and/or non-affinity based cell separation steps. In some examples, cells are washed, centrifuged, and/or incubated in the presence of one or more reagents, for example, to remove unwanted components, enrich for desired components, lyse or remove cells sensitive to particular reagents. In some examples, cells are separated based on one or more property, such as density, adherent properties, size, sensitivity and/or resistance to particular components.

In some examples, cells from the circulating blood of a subject are obtained, e.g., by apheresis or leukapheresis. The samples, in some aspects, contain lymphocytes, including T cells, monocytes, granulocytes, B cells, other nucleated white blood cells, red blood cells, and/or platelets, and in some aspects contains cells other than red blood cells and platelets.

In some embodiments, the blood cells collected from the subject are washed, e.g., to remove the plasma fraction and to place the cells in an appropriate buffer or media for subsequent processing steps. In some embodiments, the cells are washed with phosphate buffered saline (PBS). In some embodiments, the wash solution lacks calcium and/or magnesium and/or many or all divalent cations. In some aspects, a washing step is accomplished a semi-automated “flow-through” centrifuge (for example, the Cobe 2991 cell processor, Baxter) according to the manufacturer's instructions. In some aspects, a washing step is accomplished by tangential flow filtration (TFF) according to the manufacturer's instructions. In some embodiments, the cells are resuspended in a variety of biocompatible buffers after washing, such as, for example, Ca⁺⁺/Mg⁺⁺ free PBS. In certain embodiments, components of a blood cell sample are removed and the cells directly resuspended in culture media. In some embodiments, the methods include density-based cell separation methods, such as the preparation of white blood cells from peripheral blood by lysing the red blood cells and centrifugation through a Percoll or Ficoll gradient.

In some embodiments, the isolation methods of a cell population include the separation of different cell types based on the expression or presence in the cell of one or more specific molecules, such as surface markers, e.g., surface proteins, intracellular markers, or nucleic acid. In some embodiments, any known method for separation based on such markers may be used. In some embodiments, the separation is affinity- or immunoaffinity-based separation. For example, the isolation in some aspects includes separation of cells and cell populations based on the cells' expression or expression level of one or more markers, typically cell surface markers, for example, by incubation with an antibody or binding partner that specifically binds to such markers, followed generally by washing steps and separation of cells having bound the antibody or binding partner, from those cells having not bound to the antibody or binding partner.

Such separation steps can be based on positive selection, in which the cells having bound the reagents are retained for further use, and/or negative selection, in which the cells having not bound to the antibody or binding partner are retained. In some examples, both fractions are retained for further use. In some aspects, negative selection can be useful where no antibody is available that specifically identifies a cell type in a heterogeneous population, such that separation is carried out based on markers expressed by cells other than the desired population.

The separation need not result in 100% enrichment or removal of a particular cell population or cells expressing a particular marker. For example, positive selection of or enrichment for cells of a particular type, such as those expressing a marker, refers to increasing the number or percentage of such cells, but need not result in a complete absence of cells not expressing the marker. Likewise, negative selection, removal, or depletion of cells of a particular type, such as those expressing a marker, refers to decreasing the number or percentage of such cells, but need not result in a complete removal of all such cells.

In some examples, multiple rounds of separation steps are carried out, where the positively or negatively selected fraction from one step is subjected to another separation step, such as a subsequent positive or negative selection. In some examples, a single separation step can deplete cells expressing multiple markers simultaneously, such as by incubating cells with a plurality of antibodies or binding partners, each specific for a marker targeted for negative selection. Likewise, multiple cell types can simultaneously be positively selected by incubating cells with a plurality of antibodies or binding partners expressed on the various cell types.

In aspects, the cells are isolated before the muscle disease (e.g., Duchenne muscular dystrophy) begins in the subject. In other aspects, the cells are isolated after the muscle disease (e.g., Duchenne muscular dystrophy) beings in the subject.

In aspects, the cells are cultured to expand their number before being administered to the subject. In aspects, the cell population is cultured to expand their number. For example, the number may be increased by at least 0, 20, 30, 40, 50, 60, 70, 80, 90, 100, 125, 150, 175, or 200%. In embodiments, the number may increase by at least 50%, 100%, 200%, 300%, 400%, 500%, 600%, 700%, 800%, 900%, 1000%, 1100%, or 1200%. In embodiments, the cell population is cultured and expanded by at least 10%, by at least 20%, by at least 30%, by at least 40%, by at least 50%.

In one embodiment, the cells described herein are transplantable, e.g., a population of myogenic progenitor cells (MPCs) and can be administered to a subject. In some embodiments, the subject who is administered a population of cells is the same subject from whom the cell population is obtained (e.g. for autologous cell therapy). In some embodiments, the subject is a different subject. In some embodiments, a subject is suffering from a muscle injury, or is a normal subject. For example, the cells for transplantation (e.g. a composition comprising a population of myogenic progenitor cells (MPCs) can be a form suitable for transplantation.

Treatments

Compositions, uses, therapies, medicaments and methods are also provided for treating degenerative muscular wasting diseases or conditions, such as those illustrated below.

Muscular Dystrophies

Muscular dystrophies are a group of inherited diseases that cause progressive weakness and loss of muscle mass. In muscular dystrophy, abnormal genes (mutations) interfere with the production of proteins needed to form healthy muscle. For the most part, the satellite cells within muscular dystrophy patients lack the proteins necessary for muscle production. The satellite cells developed herein (e.g., the PAX7+ MPCs obtained in vitro from pluripotent stem cells) are fully functional and healthy. They can undergo asymmetric cell division and give rise to myoblasts that will fuse with the hosts myoblasts to restore damaged muscle and create new muscle. The experimental data supports that this will have a curative long term effect on continuous muscle regeneration.

Myopathies

Myopathies is a group of inherited muscle diseases associated with the loss of muscle function and strength. It is unknown what causes inflammatory myopathies, however, the belief is that something goes wrong in the immune system, which leads to an attack of the muscle cells. Causes also include infection, muscle injury due to medicine, inherited diseases that affect muscle function, disorders of electrolyte levels, and thyroid disease. Satellite cells can be designed that can produce muscle that can evade the immune system. Therefore, it is believed that the myogenic progenitor cells will not be able to be targeted by the immune system and could thus repair muscle function and strength to patients.

Mitochondrial Diseases

Mitochondrial diseases are a group of inherited diseases where the disease occurs when mitochondria fail to produce enough energy for the body to function properly. As a result, this will lead to muscle weakness, muscle pain, and low muscle tone. It is believed that the present satellite cells can significantly improve the quality of lives for patients suffering from mitochondrial diseases as they will help decrease muscle weakness by restoring muscle function and strength.

Soft Tissue Sarcomas

Soft tissue sarcomas are a group of localized soft tissue cancers. For example, Rhabdomyosarcoma (RMS), is an aggressive and highly malignant form of cancer that develops from skeletal (striated) muscle cells that have failed to fully differentiate. It is typically treated via surgery and chemotherapy and tends to yield a high survival rate. However, afterwards the patient is left with a very weak muscles in the affected areas because it is either surgically removed and or damaged by localized radiation therapy. It is believed that injections of the satellite cells could help restore muscle regeneration in the muscle areas of the patients who survive these diseases.

Ion Channel Diseases

Ion channel diseases are a group of diseases associated with defects in ion channels which are typically marked by muscular weakness, absent muscle tone, or episodic muscle paralysis. It is believed that the satellite cells can help restore functional muscle in these patients.

Cachexia

Cachexia is a complex and multifactorial disorder characterized by pathophysiological changes that alter body composition (through muscle loss), quality of life, performance status, morbidity, and mortality, with up to half of patients with cancer dying with cachexia and up to 20% of them having cachexia as the cause of death. In view of its increased prevalence, cachexia has been proposed to be a cancer comorbidity. It is believed that the satellite cells can significantly improve the quality of lives for patients suffering from cachexia as they will help restore muscle regeneration.

Sarcopenia

Sarcopenia is a condition characterized by loss of skeletal muscle mass and function. Although it is primarily a disease of the elderly, its development may be associated with conditions that are not exclusively seen in older persons. Sarcopenia is a syndrome characterized by progressive and generalized loss of skeletal muscle mass and strength and it is strictly correlated with physical disability, poor quality of life and death. The loss of satellite cells overtime is believed to be one of the leading causes that attributes to sarcopenia. It is believed that injections of the satellite cells can significantly slow the progressiveness of sarcopenia.

Non-limiting examples of degenerative muscular wasting conditions include:

Muscular Dystrophies:
Limb-girdle muscular dystrophies (LGMD)
Becker muscular dystrophy (BMD)
Congenital muscular dystrophies (CMD)
   Bethlem CMD
   Fukuyama CMD
   Muscle-eye-brain diseases (MEBs)
   Rigid spine syndromes
   Ullrich CMD
   Walker-Warburg syndromes (WWS)
Duchenne muscular dystrophy (DMD)
Emery-Dreifuss muscular dystrophy (EDMD)
Facioscapulohumeral muscular dystrophy (FSHD)
Myotonic dystrophy (DM)
Oculopharyngeal muscular dystrophy (OPMD)

Myopathies:
Congenital myopathies
   Cap myopathies
   Centronuclear myopathies
   Congenital myopathies with fiber type disproportion
   Core myopathies
   Central core disease
   Multiminicore myopathies
   Myosin storage myopathies
   Myotubular myopathy
   Nemaline myopathies
Distal myopathies
   GNE myopathy/Nonaka myopathy/hereditary inclusion-body myopathy (HTBM)
   Laing distal myopathy
   Markesberg-Griggs late-onset distal myopathy
   Miyoshi myopathy
   Udd myopathy/tibial muscular dystrophy
   VCP Myopathy/IBMPFD
   Vocal cord and pharyngeal distal myopathy
   Welandcr distal myopathy
Endocrine myopathies
   Hyperthyroid myopathy
   Hypothyroid myopathy
Inflammatory myopathies
   Dermatomyositis
   Inclusion-body myositis
   Polymyositis
Metabolic myopathies
   Acid maltase deficiency (AMD, Pompe disease)
   Carnitine deficiency
   Carnitine palmityl transferase dillciency
   Debrancher enzyme deficiency (Cori disease, Forbes disease)
   Lactate dehydrogenase deficiency
   Myoadenylate deaminase deficiency
   Phosphofructokinase deficiency (Tarui disease)
   Phosphoglycerate kinase deficiency
   Phosphoglycerate mutasc deficiency
   Phosphorylase deficiency (McArdle disease)
Myofibrillar myopathies (MFM)
Scapuloperoneal myopathy

### Mitochondrial Diseases:

Friedreich's ataxia (FA)
Mitochondrial myopathies
   Kearns-Sayre syndrome (KSS)
   Leigh syndrome (subacute necrotizing encephalomyopathy)
   Mitochondrial DNA depletion syndromes
   Mitochondrial encephalomyopathy, lactic acidosis and stroke-like episodes (MELAS)
   Mitochondrial neurogastrointestinal encephalomyopathy (MNGIE)
   Myoclonus epilepsy with ragged red fibers (MERRF)
   Neuropathy, ataxia and retinitis pigmentosa (NARP)
   Pearson syndrome
   Progressive external opthalmoplegia (PEO)

Soft Tissue Sarcomas:
Angiosarcoma
Dermatofibrosarcoma protuberans
Epithelioid sarcoma
Gastrointestinal stromal tumor (GIST)
Kaposi's sarcoma
Leiomyosarcoma
Liposarcoma
Malignant peripheral nerve sheath tumors
Myxofibrosarcoma
Pleomorphic sarcoma
Rhabdomyosarcoma
Solitary fibrous tumor
Synovial sarcoma
Undifferentiated pleomorphic sarcoma

Ion Channel Diseases:
Andersen-Tawil syndrome
Hyperkalemic periodic paralysis
Hypokalemic periodic paralysis
Myotonia congenita
   Becker myotonia
   Thomsen myotonia
Paramyotonia congenita
Potassium-aggravated myotonia

Cachexia

Sarcopenia

In embodiments, the degenerative muscular wasting disease is a muscular dystrophy or a muscle wasting disease. Exemplary muscular dystrophies include Duchenne Muscular Dystrophy, Becker Muscular Dystrophy, Limb Girdle Muscular Dystrophy, Facioscapulohumeral Muscular Dystrophy, Oculopharyngeal muscular dystrophy, Emery-Dreifuss muscular dystrophy, Fukuyama-type congenital muscular dystrophy, Miyoshi myopathy, Ullrich congenital muscular dystrophy, Steinert Muscular Dystrophy.

In embodiments, the muscular dystrophy comprises Duchenne muscular dystrophy (DMD).

The method can further include administering the cells to a subject in need thereof, e.g., a mammalian subject, e.g., a human subject. The source of the cells can be a mammal, e.g. a human. The source or recipient of the cells can also be a non-human subject, e.g., an animal model. The term “mammal” includes organisms, which include mice, rats, cows, sheep, pigs, rabbits, goats, horses, monkeys, dogs, cats, and humans. Likewise, transplantable cells can be obtained from any of these organisms, including a non-human transgenic organism. In one embodiment, the transplantable cells are genetically engineered, e.g., the cells include an exogenous gene or have been genetically engineered to inactivate or alter an endogenous gene.

A composition comprising a population of myogenic progenitor cells (MPCs) can be administered to a subject using an implantable device. Implantable devices and related technology are known in the art and are useful as delivery systems where a continuous, or timed-release delivery of compounds or compositions delineated herein is desired. Additionally, the implantable device delivery system is useful for targeting specific points of compound or composition delivery (e.g., localized sites, organs). (Negrin et al., Biomaterials, 22(6):563 (2001)). Timed-release technology involving alternate delivery methods can also be used. For example, timed-release formulations based on polymer technologies, sustained-release techniques and encapsulation techniques (e.g., polymeric, liposomal) can also be used for delivery of compounds and compositions delineated herein.

In embodiments, provided herein are methods for increasing the level of an early myogenic marker in a subject in need thereof. In further embodiments, the method comprises administering to the subject, an effective amount of the population of myogenic progenitor cells produced according to the methods described herein.

In other embodiments, the methods for treating a muscle disease or muscular dystrophies (e.g., Duchenne muscular dystrophy) comprise administering to a subject a population of myogenic progenitor cells produced according to the methods described herein, in combination with methods for controlling the outset of symptoms. In particular, the combination treatment can include administering corticosteroids (e.g., such as prednisolone and deflazacort), 02 agonists (e.g., salbutamol (e.g., albuterol). Additionally, combination therapy may include nonjarring physical activity (e.g., including, swimming), physical therapy, orthopedic appliances (such as braces and wheelchairs), appropriate respiratory support.

In embodiments, the combination therapy may include administration of the myogenic progenitor cells produced according to the methods described herein in combination with ataluren (PTC124) (IUPAC name: 3-[5-(2-Fluorophenyl)-1,2,4-oxadiazol-3-yl]benzoic acid), the structure of which is provided below:

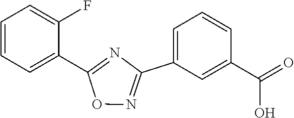 [SMILES: O=C(O)c1cccc(-c2noc(-c3ccccc3F)n2)c1]

The described cells can be administered as a pharmaceutically or physiologically acceptable preparation or composition containing a physiologically acceptable carrier, excipient, or diluent, and administered to the tissues of the recipient organism of interest, including humans and non-human animals.

The MPC population (e.g., a composition comprising an MPC population) can be prepared by resuspending the cells in a suitable liquid or solution such as sterile physiological saline or other physiologically acceptable injectable aqueous liquids. The amounts of the components to be used in such compositions can be routinely determined by those having skill in the art.

In examples, for injectable administration, the composition (e.g., a composition comprising an MPC population) is in sterile solution or suspension or can be resuspended in pharmaceutically- and physiologically-acceptable aqueous or oleaginous vehicles, which may contain preservatives, stabilizers, and material for rendering the solution or suspension isotonic with body fluids (i.e. blood) of the recipient. Non-limiting examples of excipients suitable for use include water, phosphate buffered saline, pH 7.4, 0.15 M aqueous sodium chloride solution, dextrose, glycerol, dilute ethanol, and the like, and mixtures thereof. Illustrative stabilizers are polyethylene glycol, proteins, saccharides, amino acids, inorganic acids, and organic acids, which may be used either on their own or as admixtures. The amounts or quantities, as well as the routes of administration used, are determined on an individual basis, and correspond to the amounts used in similar types of applications or indications known to those of skill in the art.

Consistent with the present invention, the MPC population can be administered to body tissues, including muscle. The number of cells in an MPC suspension and the mode of administration may vary depending on the site and condition being treated. A number of MPCs may be administered according to the invention.

In embodiments, a therapeutically effective amount of the composition (e.g., a composition comprising an MPC population) in humans can be administered. In one embodiment, the composition (e.g., a composition comprising an MPC population) is administered thrice daily, twice daily, once daily, fourteen days on (four times daily, thrice daily or twice daily, or once daily) and 7 days off in a 3-week cycle, up to five or seven days on (four times daily, thrice daily or twice daily, or once daily) and 14-16 days off in 3 week cycle, or once every two days, or once a week, or once every 2 weeks, or once every 3 weeks.

In an embodiment, the composition (e.g., a composition comprising an MPC population) is administered once a week, or once every two weeks, or once every 3 weeks or once every 4 weeks for at least 1 week, in some embodiments for 1 to 4 weeks, from 2 to 6 weeks, from 2 to 8 weeks, from 2 to 10 weeks, or from 2 to 12 weeks, 2 to 16 weeks, or longer (e.g., 1, 2, 3, 4, 5, 6, 7, 8, 9, 10, 11, 12, 36, 48, or more weeks).

### Pharmaceutical Compostions and Formulations

The present invention provides pharmaceutical compositions comprising an effective amount of a composition (e.g., a composition comprising an MPC population) and at least one pharmaceutically acceptable excipient or carrier, wherein the effective amount is as described above in connection with the methods of the invention.

In one embodiment, the composition (e.g., a composition comprising an MPC population) is further combined with at least one additional therapeutic agent in a single dosage form. In one embodiment, the at least one additional therapeutic agent comprises corticosteroids (e.g., such as prednisolone and deflazacort), 02 agonists (e.g., salbutamol (e.g., albuterol), or ataluren (PTC124) (IUPAC name: 3-[5-(2-Fluorophenyl)-1,2,4-oxadiazol-3-yl]benzoic acid).

The term “pharmaceutically acceptable” refers to those compounds, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. Examples of pharmaceutically acceptable excipients include, without limitation, sterile liquids, water, buffered saline, ethanol, polyol (for example, glycerol, propylene glycol, liquid polyethylene glycol and the like), oils, detergents, suspending agents, carbohydrates (e.g., glucose, lactose, sucrose or dextran), antioxidants (e.g., ascorbic acid or glutathione), chelating agents, low molecular weight proteins, or suitable mixtures thereof.

A pharmaceutical composition can be provided in bulk or in dosage unit form. It is especially advantageous to formulate pharmaceutical compositions in dosage unit form for ease of administration and uniformity of dosage. The term “dosage unit form” as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved. A dosage unit form can be an ampoule, a vial, a suppository, a dragee, a tablet, a capsule, an IV bag, or a single pump on an aerosol inhaler.

In therapeutic applications, the dosages vary depending on the agent, the age, weight, and clinical condition of the recipient patient, and the experience and judgment of the clinician or practitioner administering the therapy, among other factors affecting the selected dosage. Generally, the dose should be a therapeutically effective amount. Dosages can be provided in mg/kg/day units of measurement (which dose may be adjusted for the patient's weight in kg, body surface area in m², and age in years). Exemplary doses and dosages regimens for the compositions in methods of treating muscle diseases or disorders are described herein.

The pharmaceutical compositions can take any suitable form (e.g, liquids, aerosols, solutions, inhalants, mists, sprays; or solids, powders, ointments, pastes, creams, lotions, gels, patches and the like) for administration by any desired route (e.g, pulmonary, inhalation, intranasal, oral, buccal, sublingual, parenteral, subcutaneous, intravenous, intramuscular, intraperitoneal, intrapleural, intrathecal, transdermal, transmucosal, rectal, and the like). For example, a pharmaceutical composition of the invention may be in the form of an aqueous solution or powder for aerosol administration by inhalation or insufflation (either through the mouth or the nose), in the form of a tablet or capsule for oral administration; in the form of a sterile aqueous solution or dispersion suitable for administration by either direct injection or by addition to sterile infusion fluids for intravenous infusion; or in the form of a lotion, cream, foam, patch, suspension, solution, or suppository for transdermal or transmucosal administration.

In embodiments, the pharmaceutical composition comprises an injectable form.

A pharmaceutical composition can be in the form of an orally acceptable dosage form including, but not limited to, capsules, tablets, buccal forms, troches, lozenges, and oral liquids in the form of emulsions, aqueous suspensions, dispersions or solutions. Capsules may contain mixtures of a compound of the present invention with inert fillers and/or diluents such as the pharmaceutically acceptable starches (e.g., corn, potato or tapioca starch), sugars, artificial sweetening agents, powdered celluloses, such as crystalline and microcrystalline celluloses, flours, gelatins, gums, etc.

A pharmaceutical composition can be in the form of a sterile aqueous solution or dispersion suitable for parenteral administration. The term parenteral as used herein includes subcutaneous, intracutaneous, intravenous, intramuscular, intra-articular, intraarterial, intrasynovial, intrasternal, intrathecal, intralesional and intracranial injection or infusion techniques.

A pharmaceutical composition can be in the form of a sterile aqueous solution or dispersion suitable for administration by either direct injection or by addition to sterile infusion fluids for intravenous infusion, and comprises a solvent or dispersion medium containing, water, ethanol, a polyol (e.g., glycerol, propylene glycol and liquid polyethylene glycol), suitable mixtures thereof, or one or more vegetable oils. Solutions or suspensions of the compound of the present invention as a free base or pharmacologically acceptable salt can be prepared in water suitably mixed with a surfactant. Examples of suitable surfactants are given below. Dispersions can also be prepared, for example, in glycerol, liquid polyethylene glycols and mixtures of the same in oils.

The pharmaceutical compositions for use in the methods of the present invention can further comprise one or more additives in addition to any carrier or diluent (such as lactose or mannitol) that is present in the formulation. The one or more additives can comprise or consist of one or more surfactants. Surfactants typically have one or more long aliphatic chains such as fatty acids which enables them to insert directly into the lipid structures of cells to enhance drug penetration and absorption. An empirical parameter commonly used to characterize the relative hydrophilicity and hydrophobicity of surfactants is the hydrophilic-lipophilic balance (“HLB” value). Surfactants with lower HLB values are more hydrophobic, and have greater solubility in oils, while surfactants with higher HLB values are more hydrophilic, and have greater solubility in aqueous solutions. Thus, hydrophilic surfactants are generally considered to be those compounds having an HLB value greater than about 10, and hydrophobic surfactants are generally those having an HLB value less than about 10. However, these HLB values are merely a guide since for many surfactants, the HLB values can differ by as much as about 8 HLB units, depending upon the empirical method chosen to determine the HLB value.

All percentages and ratios used herein, unless otherwise indicated, are by weight. Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

### Kits for Producing Myogenic Progenitor Cells

In aspects, a kit for producing a myogenic progenitor cells is provided. In embodiments, the kit comprises a cell culture media or a cell culture medium wherein the cell culture medium is suitable for culturing a pluripotent stem cell (PSC) population.

Various alternative reagents (e.g., coatings, disassociation agents, stimulation reagents, differentiation reagents, and culture reagents such as media) may be used in embodiments herein. No specific set of reagents is required for the culturing of, e.g., PSCs, ES cells, and iPSCs. However, non-limiting examples are provided below.

In embodiments, the kit comprises a medium for growth and expansion of human iPS and hES cells. In embodiments, the medium comprises DMEM/F12, 20% knockout serum replacement, 1 mM L-glutamine, 100 mM MEM non-essential amino acids, and 0.1 mM 0-mercaptoethanol. In embodiments, 10 ng/mL of FGF-2 may be added after sterile filtration.

In embodiments, the kit comprises a reagent for ES and/or iPS cell selection and/or passaging. In embodiments, the reagent is ReLeSR™ passaging reagent (Stemcell Technologies, Vancouver Canada, Catalog No. 05872 or 05873). In embodiments, the reagent is mTeSR™1 (Stemcell Technologies, Vancouver Canada, Catalog No. 85850 or 85857). In embodiments, the reagent is Vitronectin XF™ (Stemcell Technologies, Vancouver Canada, Catalog No. 07180 or 07190). In embodiments, the reagent is Gentle Cell Dissociation Reagent (Stemcell Technologies, Vancouver Canada, Catalog No. 07174). Many plate coating reagents and ES/iPSC medium are commercially available and will be known to those skilled in the art. In embodiments, any plate coating reagent may be used. In embodiments, the coating reagent is GelTrex from Gibco (Invitrogen, A1413302). In other embodiments, the cells may be passaged weekly, using 6 U/mL dispase (Invitrogen, 17105041) or mechanically.

In embodiments, the kit comprises a reagent comprising one or more cell-dissociation enzymes. In embodiments, the reagent is TrypLE™ cell dissociation reagent (ThermoFisher Catalog No: A1285901). In embodiments, the reagent is TrypLE™ Express (Thermo Fisher SKU No. 12604-013). In embodiments, the reagent is StemPro™ Accutase™ Cell Dissociation Reagent (Thermo Fisher Catalog No. A1110501). Various disassociation reagents are known in the art and may be used. In embodiments, cells may be physically scraped off a culture surface (such as a plate). In embodiments, the dissociation reagent includes Trypsin EDTA 0.25% Trypsin with EDTA 4Na 1×(Invitrogen, 25200114); or in other embodiments, Accutase (Invitrogen, S-1100-1, AT-104). In embodiments, the kit comprises basic fibroblast growth factor (FGF2) and/or fibroblast growth factor 8.

In embodiments, a cell culture medium in the kit is suitable for culturing a PSC population.

In embodiments, a cell culture medium in the kit comprises about 5%, 4.5%, 4%, 3.5%, 3%, 2.5%, 2%, 1.5%, 1%, or 0.5% serum. In embodiments, the kit is configured for use of about 5%, 4.5%, 4%, 3.5%, 3%, 2.5%, 2%, 1.5%, 1%, or 0.5% serum or less.

In embodiments, a cell culture medium in the kit is a serum-free cell culture medium.

In embodiments, the kit does not comprise serum.

The present invention also provides packaging and kits comprising pharmaceutical compositions for use in the methods of the present invention. The kit can comprise one or more containers selected from the group consisting of a bottle, a vial, an ampoule, a blister pack, and a syringe. The kit can further include one or more of instructions for use in treating and/or preventing a disease, condition or disorder of the present invention (e.g., a muscular dystrophy), one or more syringes, one or more applicators, or a sterile solution suitable for reconstituting a pharmaceutical composition of the present invention.

### EXAMPLES

The following examples illustrate certain specific embodiments of the invention and are not meant to limit the scope of the invention.

Embodiments herein are further illustrated by the following examples and detailed protocols. However, the examples are merely intended to illustrate embodiments and are not to be construed to limit the scope herein. The contents of all references and published patents and patent applications cited throughout this application are hereby incorporated by reference.

### Example 1: PAX7::MPC Derived from Human Pluripotent Stem Cells (hPSCs) could be Maintained Ex Vivo and Participate in Muscle Regeneration

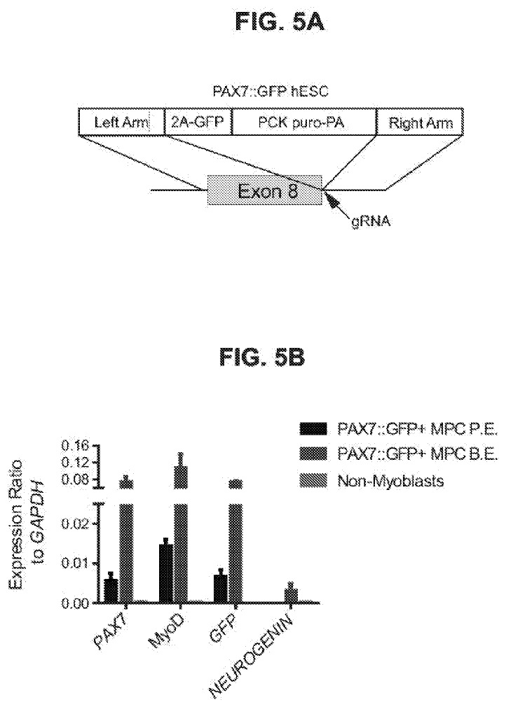

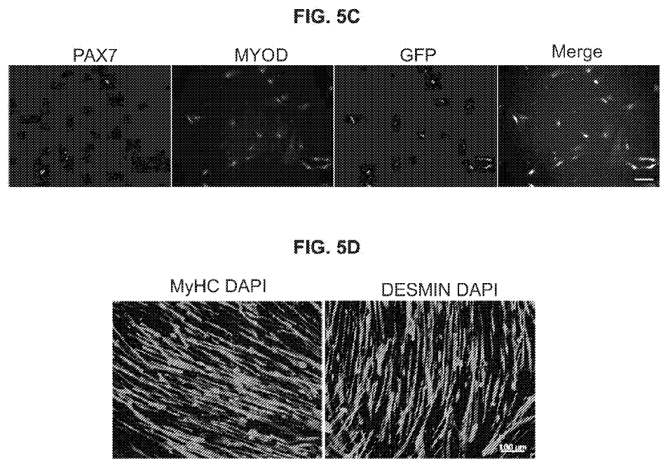

PAX7 is specifically expressed in myogenic lineages during muscle development and in adult muscle stem cells⁷. Due to the self-renewal capability, mouse muscle stem cells, compared with the myoblasts, have achieved more desirable transplantation results^{2,8}. In order to obtain the progenitor cell population to study the potential muscle stem cell transplantation therapy in humans, a PAX7::GFP hESC reporter cell line was obtained by employing CRISPR-Cas9 technique (FIG. 5A).

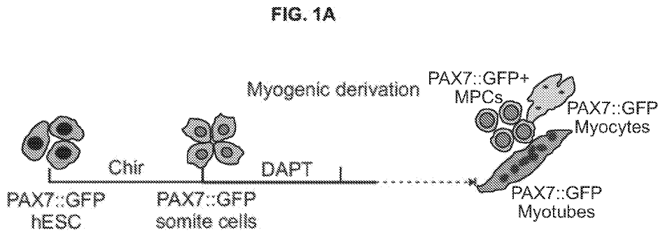

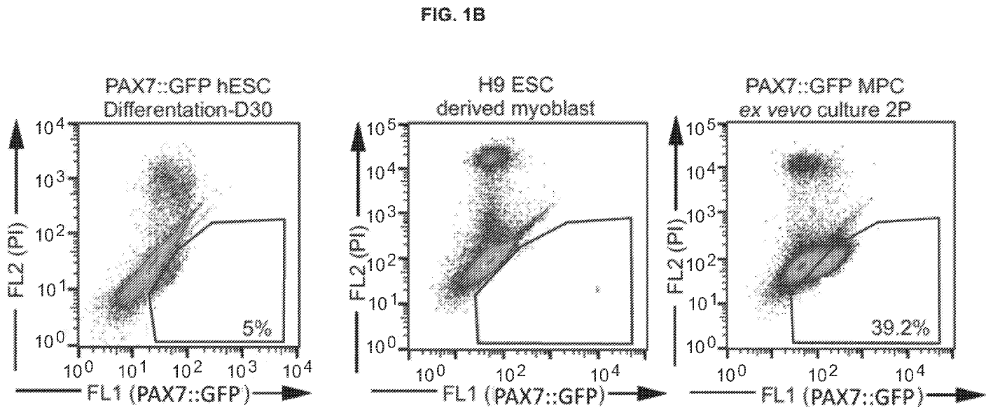

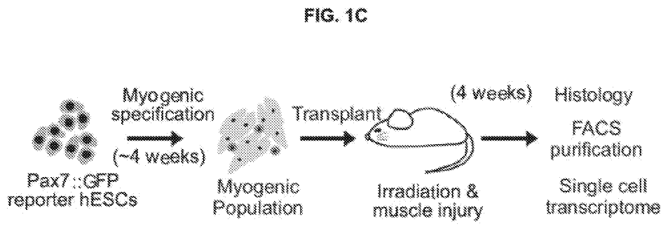

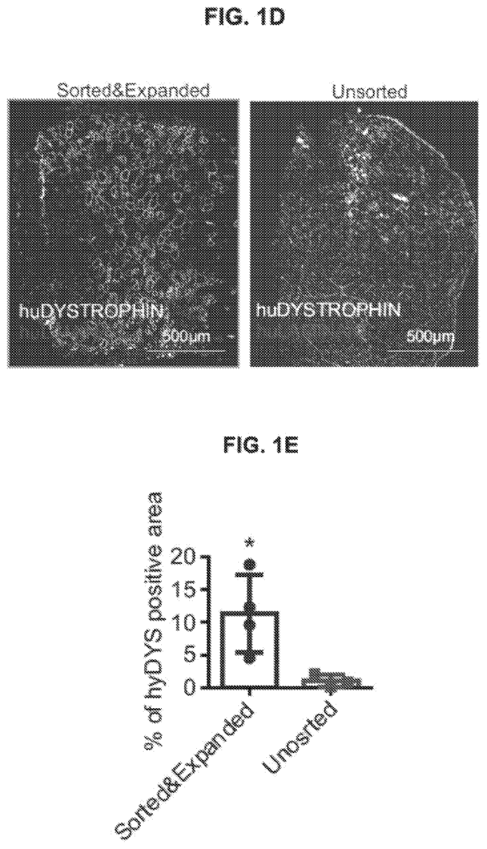

During the myogenic derivation process from hPSCs 6 (FIG. 1A), PAX7::GFP expressing cells can be detected on day 18 and cell number gradually increased between day 18 to day 30. When harvested on day 30, the percentage of GFP expressing cells can reach 5-10% (FIG. 1B). After being isolated by FACS and maintained ex vivo in culture, they expanded rapidly and around 30-40% of the cells remained GFP expression detected by flowcytometer and IF when cultured in medium with the addition of basic-fgf2 and fgf8 (FIG. 1B and FIG. 5C). These cells are referred to as PAX7::GFP myogenic progenitor cells (PAX7::GFP MPC). In order to confirm the myogenic capacity of the PAX7::GFP MPC, it was observed that GFP⁺ cells also express the early myogenic markers PAX7 and/or MYOD in PAX7::GFP MPC ex vivo culture (FIG. 5B-C). And these PAX7::GFP MPC were able to fuse into myotubes after medium serum withdrawal (FIG. 5C).

### Example 2: Myogenic Regeneration Ability of PAX7::GFP MPCs

In order to examine the myogenic regeneration ability of these cells, unsorted cells derived directly from Pax7::GFP hESC myogenic derivation culture (unsorted) as well as Pax7::GFP MPCs expanded ex vivo (sorted&expanded) were transplanted into immunodeficient mice (NSG) to compare the transplantation schemes (FIG. 1B). One tibialis anterior (TA) of these mice were irradiated to deplete the satellite cells and muscle fibers were damaged by 1.2% BaCl₂ to allow de novo fiber forming from transplanted cells. TAs were harvest 4 weeks post transplantation (FIG. 1C). The sorted and expanded PAX7::GFP MPCs differentiated and fused into myofibers detected by human specific DYSTROPHIN antibody (FIGS. 1D-1E). On the contrary, the unsorted cells failed to regenerate into organized myofibers, instead, the majority of the cells did not form myotubes and possibly trans-differentiated into fibroblasts (FIGS. 1D-1E). Only small and limited amount DYSTROPHIN positive myofibers were detected which has been the limiting factor prohibiting the clinical success of muscle stem cell therapy⁹⁻¹³. The cells may have been dissociated directly from culture without purification, and therefore may have contained other cell types such as neurons and fibroblasts.¹⁴

In the following studies, sorted and expanded PAX7::GFP⁺ MPC cells were used. The above evidence supports that PAX7::GFP MPC could participate in muscle regeneration and fuse into host muscle forming huDYSTROPHIN expressing myofibers.

### Example 3: Repopulation and Engraftment Analysis of PAX7::GFP MPCs and Analysis of their Myogenic Progenitor Property

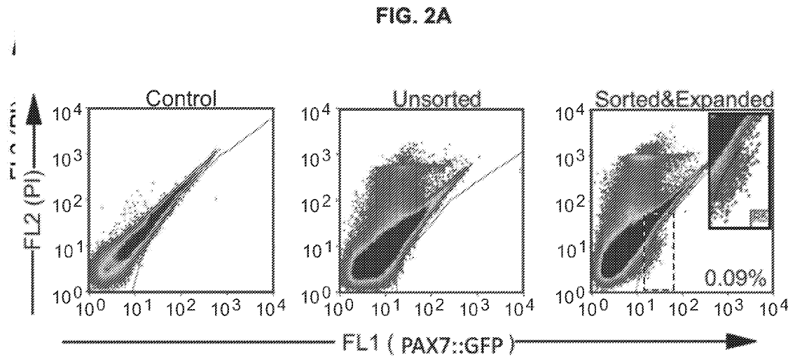

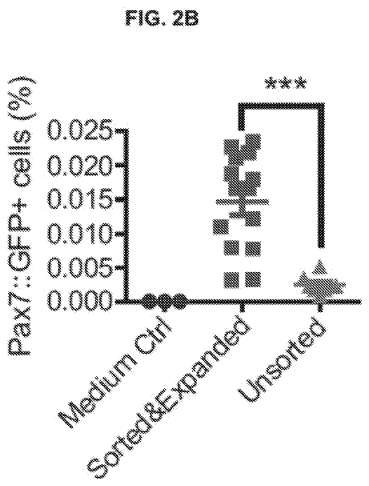

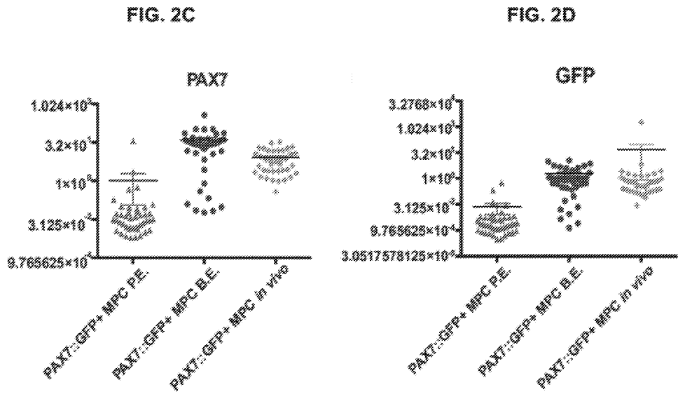

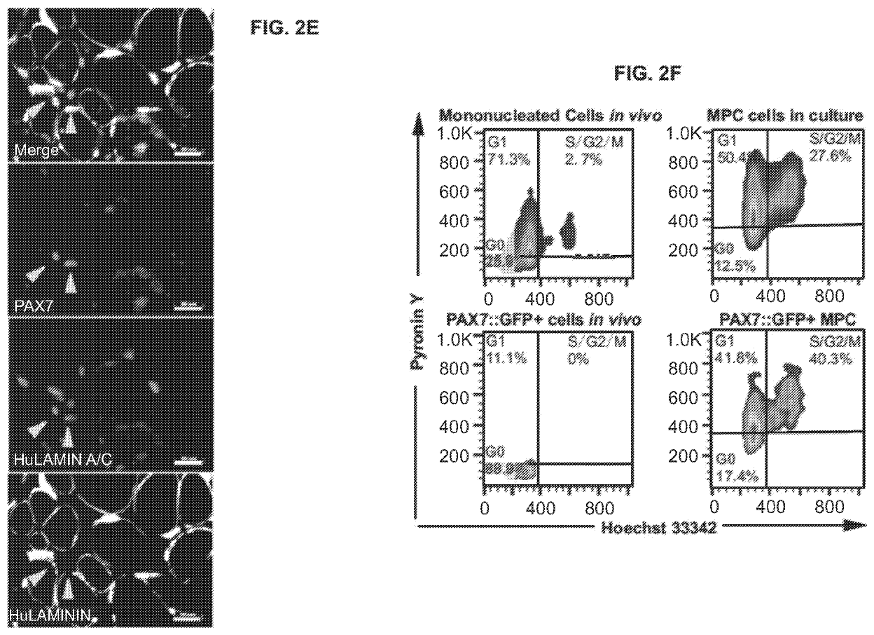

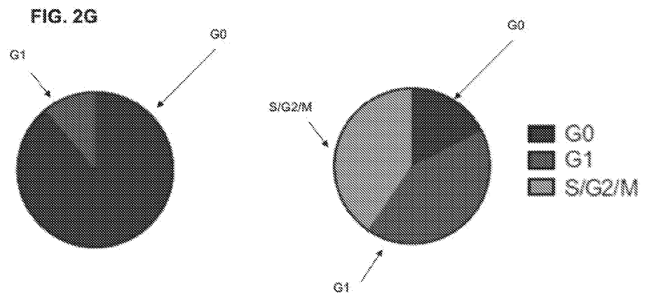

To better understand the repopulation and engraftment ability of PAX7::GFP MPCs and to further confirm their myogenic progenitor property, the fate of transplanted MPCs with GFP signal was traced after in vivo transplantation into TA of NSG mice. Four weeks post the transplantation, cells were isolated from TA and mononucleated cells were collected. The percentage of cells still expressing GFP four weeks post transplantation that recovered from the total number of mononucleated cells can reach up to 0.025% when isolated from single TA or 0.09% when 4 TAs were polled together (FIGS. 2A and 2B). To be assured of the status of PAX7::GFP MPC isolated from TA (PAX7::MPC in vivo), the single-cell QPCR detected the expression level of PAX7 and GFP in the isolated GFP expressing single cells from TA was higher compared with PAX7::GFP⁺ MPCs before transplantation (Post Expansion: P.E.) similar to the level before expansion (B.E.) (FIGS. 2C-2D).

Next, the location of PAX7::GFP⁺ cells was examined in vivo. PAX7 and huLAMINA/C double labelling were used to trace the progenies of PAX7::GFP⁺ MPCs. Among all the human cells, some of the cells were found to reside under the basal lamina where adult muscle stem cells locate (FIG. 2E). And when these cells were isolated, the cells that were PAX7::GFP positive were detected to be at GO phase of the cell cycle which is the character of quiescent stem cells (FIGS. 2F-2G).

In summary, the transplanted PAX7::GFP MPCs participated in muscle regeneration as well as becoming PAX7 expressing cells harboring in the muscle stem cell niche area which is referred to as engrafted PAX7::GFP⁺ MPCs.

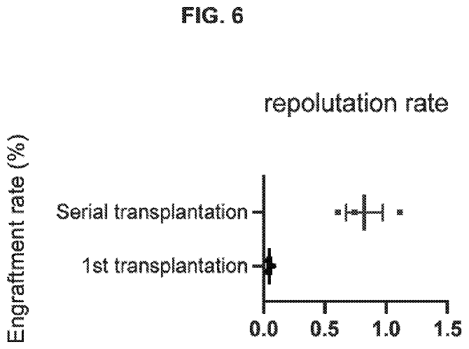

This example then further quantified the percentage of cells that located in the niche area post transplantation against the cell number used for transplantation from the IF sections. The cells were expanded in vitro and were transplanted in mice. Four weeks later the cells were isolated from these primary mice and transplanted into twenty other mice. The results (FIG. 6) demonstrate that the cells can efficiently engraft in vivo. The percentage of cells that located in the niche area were quantified post transplantation against the cell number used for transplantation from the IF sections.

### Example 4: Analysis of PAX7::GFP+ Engrafted Cells and the “Adult Satellite Cell” Property

Next it was determined whether PAX7::GFP⁺ engrafted cells had the “adult satellite cells” property. Single cell RNA-seq was used to analyze and interpret the transcriptional profile change during the transition process of PAX7::GFP MPC become PAX7::GFP engrafted cells. Meanwhile, the undifferentiated OCT4::GFP⁺ hESCs and expanded Pax7::GFP MPC in vitro were included for a month.

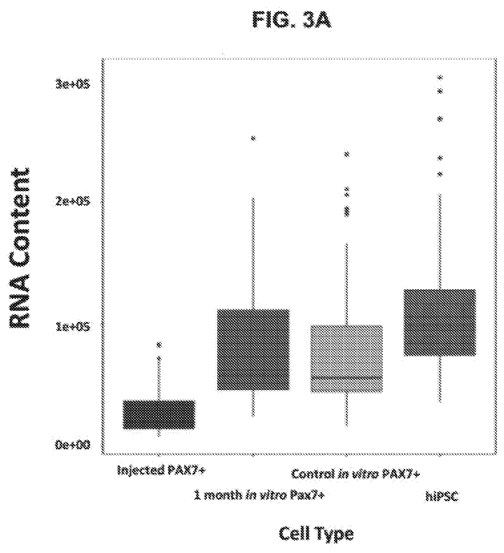

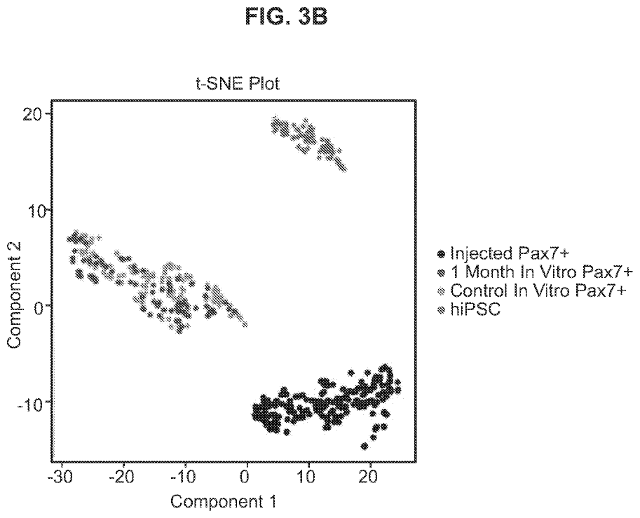

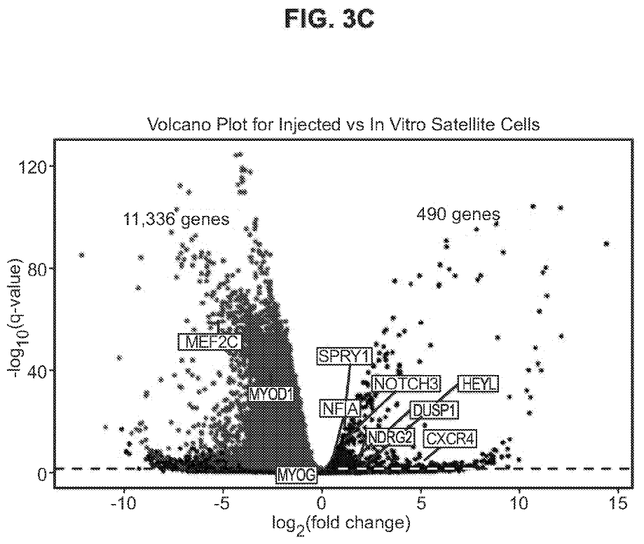

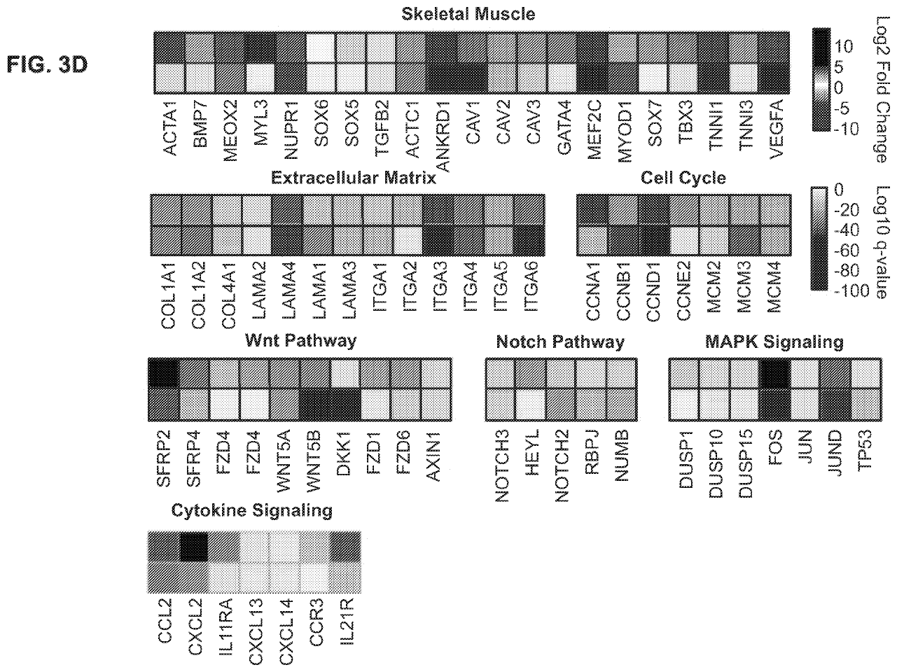

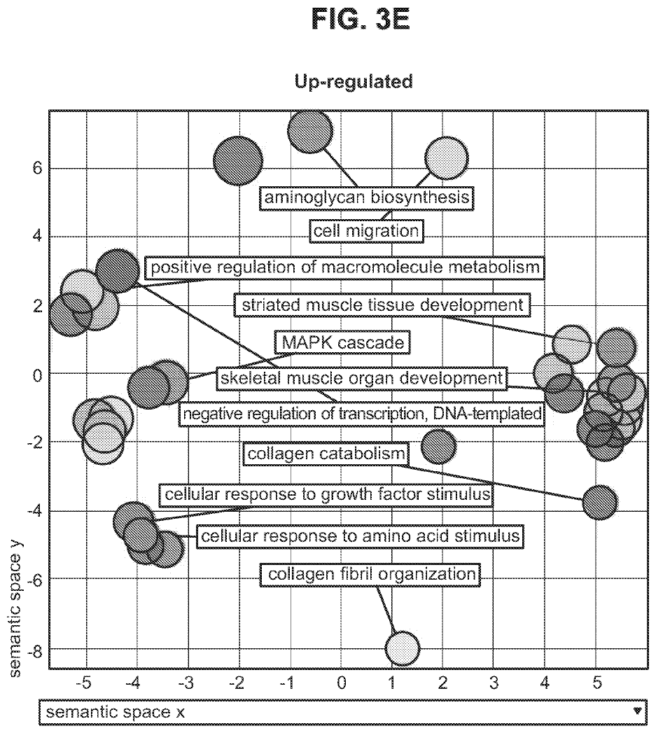

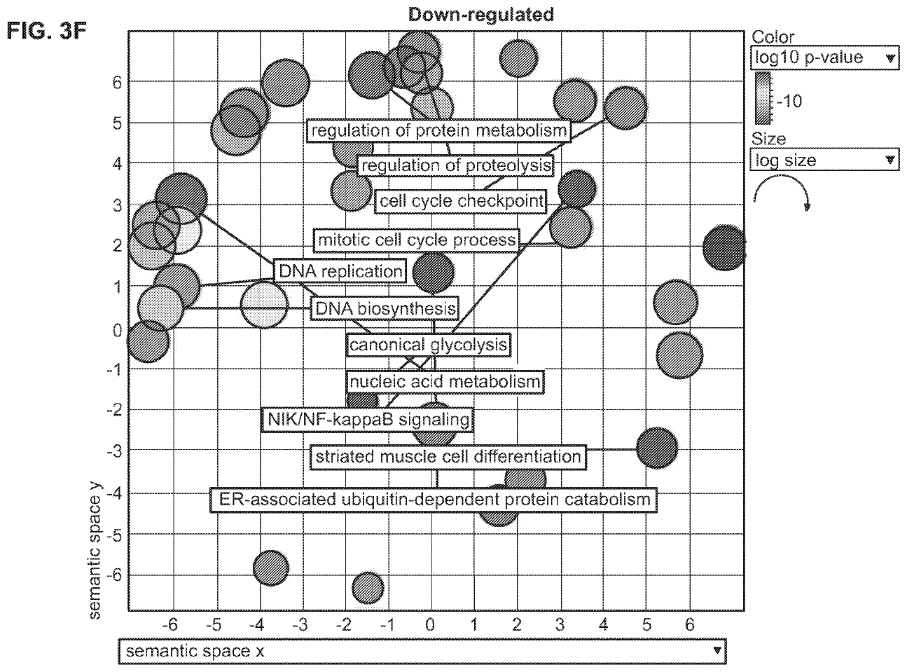

First it was noticed that the Pax7GFP⁺ engrafted cells had lower RNA content than Pax7GFP MPC (FIG. 3A). This phenomenon was found in quiescent satellite cells when compared to activated satellite cells^{15,16}. The principal component analysis (PCA) demonstrated the hESCs, injected and expanded cells (1 week and 1 month in vitro expansion) were tightly clustered together with their own type of cells. Another observation from PCA was that 1 month expansion ex vivo did not alter the PAX7::GFP MPC property transcription-wise.

In order to understand the change that took place during the PAX7::GFP engraftment process, the transcription profile change was compared between PAX7::GFP MPC and the engrafted PAX7::GFP MSCs. In the engrafted PAX7::GFP MSCs compared with PAX7::GFP⁺ MPCs 490 genes were upregulated and 11,336 genes were down-regulated (FIG. 3C). Genes that regulate cell cycle progression were downregulated indicating that engrafted cells exited cell cycle which was observed in mouse satellite cells in vivo. Among the down regulated genes, MyoD1 which was reported to be lost when satellite cells returning to quiescent status¹⁷ was found to be suppressed in the engraftment process. Another two transcription factors MEF2C and Myogenin whose expression marked the muscle stem cell differentiation decreased their expression¹⁸. Transcripts that were highly upregulated include extracellular matrix related genes (COL1A1 and LAMA4) and notch signaling related genes (NOTCH3 and HEYL) that negatively regulate cell differentiation (FIG. 3D).

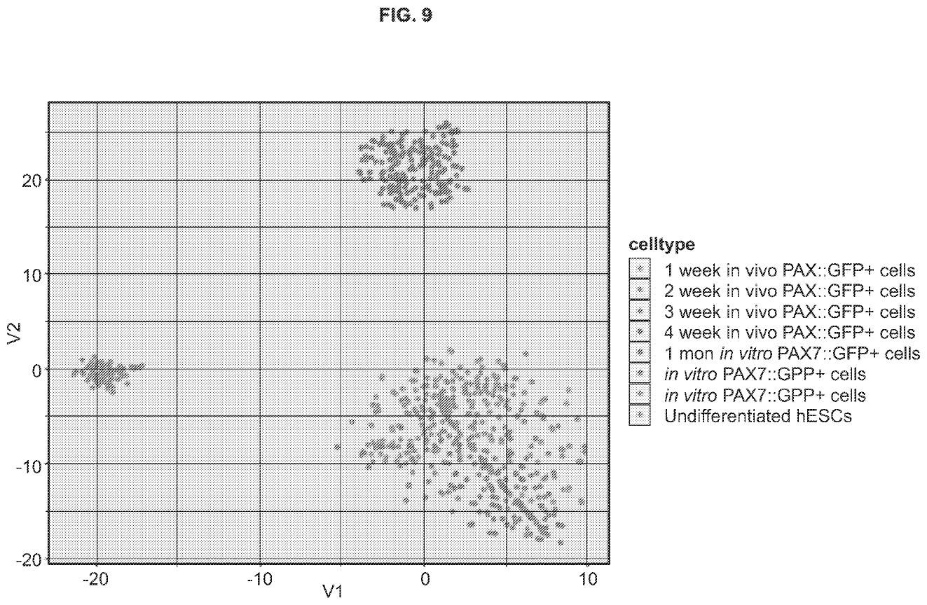

To further understand the time-line of transition to quiescence, this example performed a single cell RNA-seq on PAX7::GFP+ cells isolated from TAs at 1-, 2-, 3-, and 4-week time point following transplantation respectively. Using scran, this example sought to compare the results of our two sequencing studies. Interestingly, it was found that transcriptomic changes in the PAX7::GFP+ cells happened within the first week of transplantation and did not alter much after the first week (FIG. 9). This phenomenon suggests that the transition of PAX7:GFP+ cells to quiescent muscle stem cell status occurs relatively rapidly compared to muscle repair.

### Example 5: Analysis of PAX7::GFP+ Engrafted Cells to Self-Renew

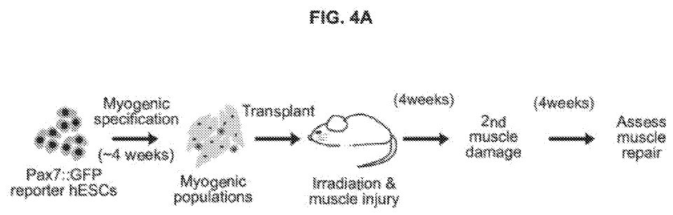

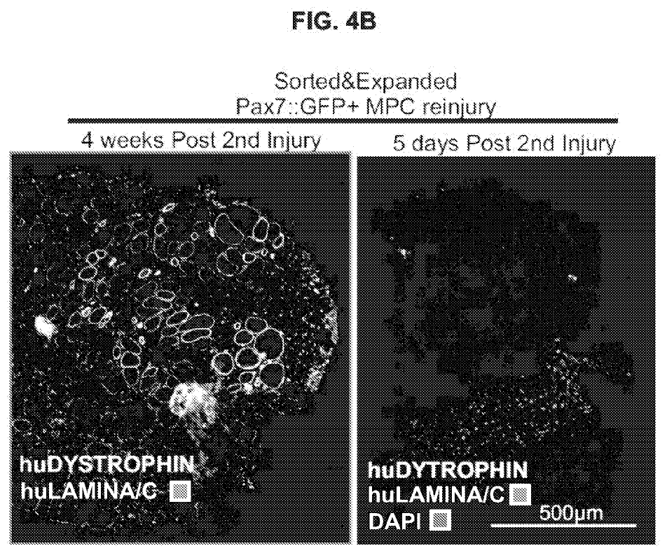

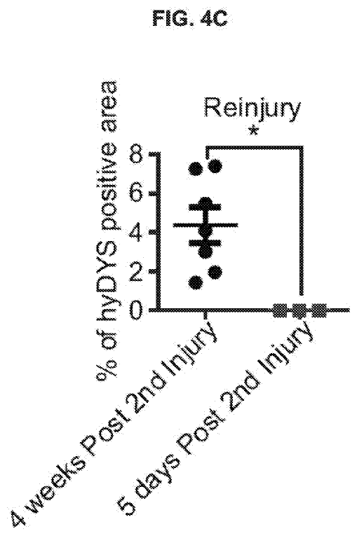

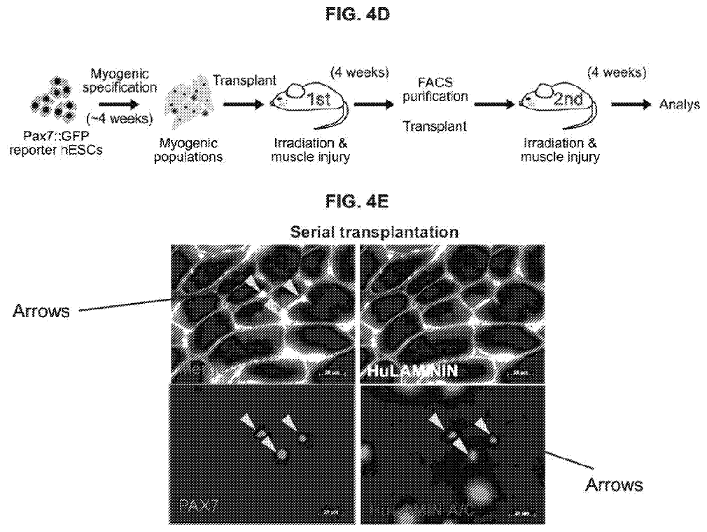

Another characteristic of muscle stem cell is their ability to self-renew which is the key to the functional engraftment of the stem cells. A reinjury experiment was first performed where TA following the first PAX7::GFP⁺ MPC transplantation was re-injured 4 weeks after the first injury (FIG. 4A). Following the second-time injury, there was no muscle fiber of human origin that formed after the first injury were left (FIG. 4B). And 4 weeks post the reinjury, robust de novo muscle regeneration was observed from progenies of PAX7::MPCs labeled by huDYTROPHIN indicating the engrafted PAX7::GFP MPCs could participate in regeneration forming muscle (FIG. 4B). Also, if engrafted PAX7::GFP MPCs cells were isolated and transplanted into second recipient mouse these cells could form myofibers as well as become PAX7 expressing cells residing in niche area in the similar manner to adult muscle stem cells (FIG. 4D-E).

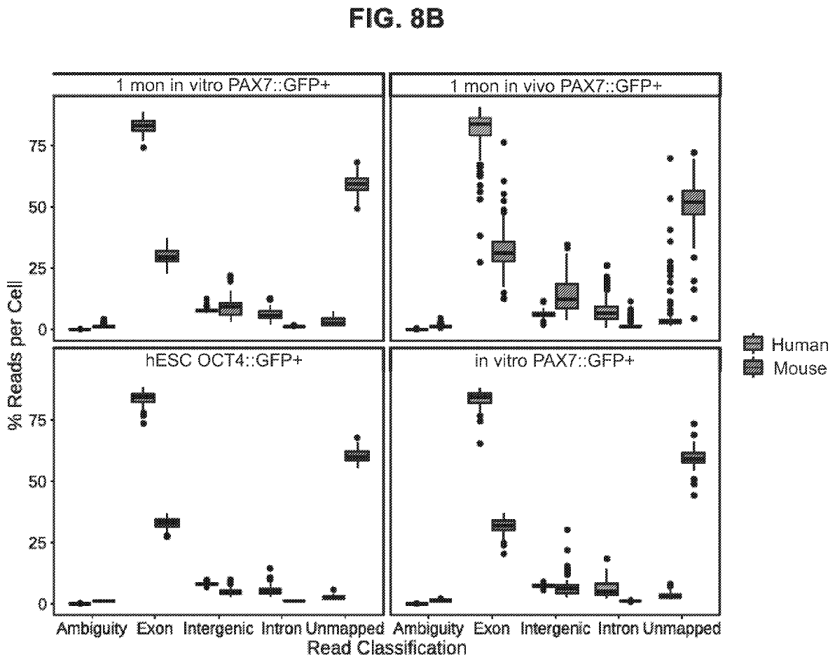

Confirmation of the human origin of the PAX7::GFP⁺ cells post transplantation is further demonstrated in FIGS. 8A and 8B. FIGS. 8A and 8B show a blot and a series of graphs demonstrating the human origin of the PAX7::GFP⁺ cells post-transplantation. FIG. 8A: genomic PCR was conducted using human specific primers to determine the origin of PAX7::GFP⁺ cells isolated from injected TA. FIG. 8B: Sequence comparison of single cell RNA-seq result shows that the majority of the PAX7::GFP⁺ cells isolated from injected TA are human cells.

### Example 6: Animal Exercise Testing

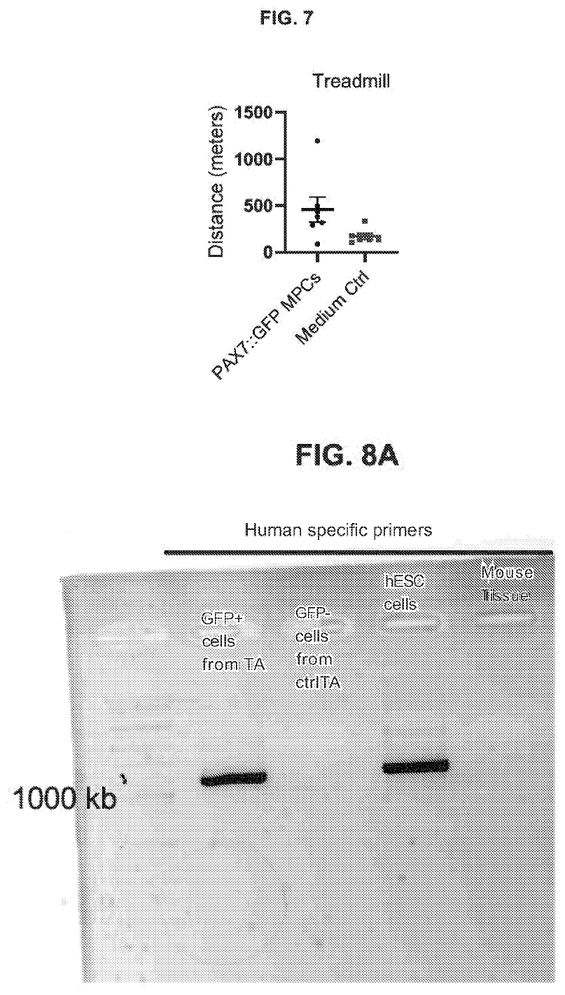

In this example, PAX7::GFP MPCs were injected into NSG-MDX mice (DMD mouse model with the compromised immune-system). Treadmill running tests were then performed. As shown in FIG. 7, the results show an increase in these mice improvement as compared to the control.

### Example 7: Characterization of Expanded PAX7::GFP⁺ MPC Cells

The above examples have demonstrated that the PAX7::GFP⁺ MPC cells derived from hPSC have excellent repopulation and engraftment abilities. This example characterized the cells with respect to their molecular signatures, in particular cell surface markers.

Single cell RNA expression analysis revealed a unique signature of cell surface markers for these PAX7::GFP⁺ MPC cells. The signature included CHRNA1+, NTSR1+, FZD1+, FZD5−, GPR37− and GPR27−, as summarized in the table below.

| Expression | Cell surface marker | Name |
|---|---|---|
| Positive | CHRNA1 | Cholinergic receptor nicotinic alpha 1 |
| Positive | NTSR1 | Neurotensin receptor 1 |
| Positive | FZD1 | Frizzled class receptor 1 |
| Negative | FZD5 | Frizzled class receptor 5 |
| Negative | GPR37 | G protein-coupled receptor 37 |
| Negative | GPR27 | G protein-coupled receptor 27 |

Example 2 demonstrated that sorted and expanded PAX7::GFP MPCs were able to differentiate and fuse into myofibers in vivo, while the unsorted cells failed to regenerate into organized myofibers. This example further explored the minimum PAX7+ cell concentration for engraftment.

When there were only about 5% or 10% PAX7+ cells in a cell population, the population had a neglectable activity in engraftment. When the PAX7+ population was at least 30%, and more apparently at 35%-40%, it exhibited efficient engraftment and tissue regeneration capabilities.

### REFERENCES

1. Mauro, A. Satellite cell of skeletal muscle fibers. The Journal of biophysical and biochemical cytology 9, 493-495 (1961).
2. Sacco, A., Doyonnas, R., Kraft, P., Vitorovic, S. & Blau, H. M. Self-renewal and expansion of single transplanted muscle stem cells. Nature 456, 502-506, doi:10.1038/nature07384 (2008).
3. Xu, X. et al. Human Satellite Cell Transplantation and Regeneration from Diverse Skeletal Muscles. Stem cell reports 5, 419-434, doi:10.1016/j.stemcr.2015.07.016 (2015).
4. Templin, C. et al. Ex vivo expanded hematopoietic progenitor cells improve cardiac function after myocardial infarction: role of beta-catenin transduction and cell dose. Journal of molecular and cellular cardiology 45, 394-403, doi:10.1016/j.yjmcc.2008.06.010 (2008).
5. Chal, J. et al. Differentiation of pluripotent stem cells to muscle fiber to model Duchenne muscular dystrophy. Nat Biotechnol 33, 962-969, doi:10.1038/nbt.3297 (2015).
6. Choi, I. Y. et al. Concordant but Varied Phenotypes among Duchenne Muscular Dystrophy Patient-Specific Myoblasts Derived using a Human iPSC-Based Model. Cell reports 15, 2301-2312, doi: 10.1016/j.celrep.2016.05.016 (2016).
7. Relaix, F. & Zammit, P. S. Satellite cells are essential for skeletal muscle regeneration: the cell on the edge returns centre stage. Development 139, 2845-2856, doi:10.1242/dev.069088 (2012).
8. Gussoni, E. et al. Dystrophin expression in the mdx mouse restored by stem cell transplantation. Nature 401, 390-394, doi:10.1038/43919 (1999).
9. Huard, J. et al. Human myoblast transplantation: preliminary results of 4 cases. Muscle & nerve 15, 550-560, doi:10.1002/mus.880150504 (1992).
10. Karpati, G. et al. Myoblast transfer in Duchenne muscular dystrophy. Annals of neurology 34, 8-17, doi:10.1002/ana.410340105 (1993).
11. Tremblay, J. P. et al. Results of a triple blind clinical study of myoblast transplantations without immunosuppressive treatment in young boys with Duchenne muscular dystrophy. Cell transplantation 2, 99-112 (1993).
12. Mendell, J. R. et al. Myoblast transfer in the treatment of Duchenne's muscular dystrophy. The New England journal of medicine 333, 832-838, doi:10.1056/NEJM199509283331303 (1995).
13. Morandi, L. et al. Lack of mRNA and dystrophin expression in DMD patients three months after myoblast transfer. Neuromuscular disorders: NMD 5, 291-295 (1995).
14. Kim, J. et al. Expansion and Purification Are Critical for the Therapeutic Application of Pluripotent Stem Cell-Derived Myogenic Progenitors. Stem cell reports 9, 12-22, doi:10.1016/j.stemcr.2017.04.022 (2017).
15. Fukada, S. et al. Molecular signature of quiescent satellite cells in adult skeletal muscle. Stem cells 25, 2448-2459, doi:10.1634/stemcells.2007-0019 (2007).
16. Rodgers, J. T. et al. mTORC1 controls the adaptive transition of quiescent stem cells from GO to G(Alert). Nature 510, 393-396, doi:10.1038/nature13255 (2014).
17. Zammit, P. S. et al. Muscle satellite cells adopt divergent fates: a mechanism for self-renewal? The Journal of cell biology 166, 347-357, doi:10.1083/jcb.200312007 (2004).
18. Ridgeway, A. G., Wilton, S. & Skerjanc, I. S. Myocyte enhancer factor 2C and myogenin upregulate each other's expression and induce the development of skeletal muscle in P19 cells. J Biol Chem 275, 41-46 (2000).

### Example 8: Materials and Methods

### Animal and Cell Transplantation Strategy

All animal experiments were approved by the Institutional Animal Care and Use Committee (IACUC) of The Johns Hopkins University, School of Medicine (Baltimore, Md.). NSG (NOD.Cg-Prkdcscid Il2rgtm1Wjl/SzJ, Jackson Lab) mice were maintained in a 12-h light cycle (7 am-7 pm) with ad libitum access to food and water. Left TA of mice were irradiated (18 g) 72 hours prior to 50 μl 1.2% BaCl₂ injury. 1 million PAX7::GFP MPCs were resuspended in 50 μl culture medium supplemented with rock inhibitor and injected into TA.

### Myogenic Cell Derivation and Cell Culture:

For myogenic linage derivation, hPSCs (H9 cell line (a human embryonic stem cell line, purchased from Coriell) and GM01582iPSC (a reprogrammed human induced pluripoitent stem cell line, with the original fibroblast line was purchased from Coriell) were plated as single cells on Geltrex (Gibco) treated dishes, at a density of 1.5×10⁵ cells per well in a 24-well plate, in the presence of MEF-conditioned N2 media containing 10 ng/ml of FGF-2 (PeproTech) and 10 μM of Y-27632 (Cayman). The cells were induced to differentiate into myoblasts by adding a selective inhibitor of glycogen synthase kinase 3 (GSK-3) e.g. CHIR99021 (Tocris, a Bio-Techne Corporation, Minneapolis, Minn.) (3 μM) in N2 medium for 4 days and by DAPT (10 μM) for the following 8 days. Cells continued to differentiate and mature in N2 medium for the next 13 days. Myoblasts were isolated by FACS with the selection marker NCAM⁺/HNK1⁻ (NCAM: 5.1H11, DSHB; HNK1: C6680, Sigma). PAX7::GFP⁺ cells were isolated by FACs with GFP signal.

The NCAM⁺/HNK1⁻ myoblasts were maintained in a humidified incubator containing 5% CO₂ at 37° C. and grown in N2 media supplemented with 10% FBS. PAX7::GFP MPCs were maintained in N₂ medium supplemented with 20% FBS, b-FGF2 and FGF8. To induce myotube formation, expanded myogenic cells were plated to confluence, and switched to N₂ media without serum.

Isolation of Mononuclear Cells from Muscle and Pyronin Hoechst Cell Cycle Analysis:

Mononuclear cells were isolated from TA muscle with PBS containing 2 mg/ml Collagenase A (Roche), 2.4 U/ml Dispase II (Roche), 10 μg/ml DNase I (Roche) and 0.38 mM CaCl₂). The digested cells were resuspended in PBS containing 2% FBS and DNaseI for FACs analysis. For cell cycle analysis, cells were fixed in 1% PFA for 15 minutes at 37° C. and permeabilized in 100% methanol over night at −20° C. The next day, cells were washed and blocked in blocking buffer (0.75% Saponin, 1% FBS, 1% BSA in PBS). Primary GFP antibody (Invitrogen) and secondary antibody (goat anti chicken 488) was stained for 30 mins at room temperature. Then cells were suspended in blocking buffer containing Hoechst 33342 (2 μg/mL) and Pyronin-Y (4 μg/mL) for 30 mins for DNA and RNA binding. Finally, cells were suspended in FACs buffer containing Pyronin-Y (2 μg/mL) for flow cytometry analysis.

### Immunocytochemistry and Immunohistochemistry

Muscle pieces were collected and frozen using −60° C. isopentane. Frozen muscle were cryosectioned into 8 μm sections. Cryosections were fixed with cold methanol, washed with PBS, and blocked in 20% normal goat serum (Vector Laboratories) and 2% bovine serum albumin (Sigma Aldrich). Primary antibodies against human lamin A+C (Mouse IgG2b, 1:100, Leica), pan-species Pax7 (IgG1, 1:1, DSHB), and human laminin (IgG2a, 1:100, DSHB) were incubated on the slides at 4° C. overnight. To detect dystrophin, a combination of primary antibodies against human dystrophin (Millipore, 1:200) and human lamin A+C were incubated on the slides at 4° C. overnight, followed by incubation with mouse anti-mouse secondary antibodies at room temperature for 1 hour. Sections were mounted using Vecta Shield mounting medium with DAPI (Vector Laboratories), and imaged using fluorescent microscopy (Zeiss).

### Single Cell RNA Sequencing

Pax7-GFP⁺ cells were sorted by Fluorescence-Activated Cell Sorting as single cells in 96-well or 384-well capture plates using a Sony SH800 sorter. Capture plate wells contained 5 μl of capture solution (1:500 Phusion High-Fidelity Reaction Buffer, New England Biolabs; 1:250 RnaseOUT Ribonuclease Inhibitor, Invitrogen). Single cell libraries were then prepared using the previously described SCRB-seq protocol¹⁻². Briefly, cells were subjected to proteinase K treatment followed by RNA desiccation to reduce the reaction volume. RNA was subsequently reverse transcribed using a custom template-switching primer as well as a barcoded adapter primer. The customized SCRB-seq barcode primers contain a unique 6 base pair cell-specific barcode as well as a 10 base pair unique molecular identifier (UMI). Transcribed products were pooled and concentrated, with unincorporated barcode primers subsequently digested using Exonuclease I treatment. cDNA was PCR-amplified using Terra PCR Direct Polymerase (Takara Bio). Final libraries were prepared using 1 ng of cDNA per library with the Nextera XT kit (Ilumina) using a custom P5 primer as previously described. Pooled libraries were sequenced on two high-output lanes of the Illumina NextSeq500 with a 16 base pair barcode read, 8 base pair i7 index read, and a 66 base pair cDNA read design.

To analyze sequencing data, reads were mapped and counted using zUMIs 2.2.33 with default settings and barcodes provided as a list. zUMIs utilizes STAR (2.5.4b)⁴ to map reads to an input reference genome and feature Counts through Rsubread (1.28.1) to tabulate counts and UMI tables. GRCh38 from Ensembl concatenated with ERCC spike-in references was used for the reference genome and gene annotations. Dimensionality reduction and cluster analysis were performed with Seurat (2.3.4)⁶, while differential gene expression analysis was done in Monocle (2.4.0)⁷. GO Enrichment Analysis was performed on differentially expressed genes through the Gene Ontology Consortium tool⁸⁻¹⁰, and visualized enriched terms and pathways with REVIGO¹¹.

### REFERENCES

1. Soumillon M, Cacchiarelli D, Semrau S, van Odenaarden A, Mikkelsen T S. Characterization of directed differentiation by high-throughput single-cell RNA-Seq. BioRxiv 2014.
2. Ziegenhain C, Vieth B, Parekh S, Reinius B, Guillaumet-Adkins A, Smets M, Leonhardt H, Heyn H, Hellmann I, Enard W. Comparative Analysis of Single-Cell RNA Sequencing Methods. Mol Cell 2017, 65(4):631-643.
3. Parekh S, Ziegenhain C, Vieth B, Enard W, Hellmann I. ZUMIs—A fast and flexible pipeline to process RNA sequencing data with UMIs. Gigascience 2018, 7(6).
4. Dobin A, Davis C A, Schlesinger F, Drenkow J, Zaleski C, Jha S, Batut P, Chaisson M, Gingeras T R. STAR: ultrafast universal RNA-seq aligner. Bioinformatics 2013, 29(1):15-21.
5. Liao Y, Smyth G K, Shi W. The Subread aligner: fast, accurate and scalable read mapping by seed-and-vote. Nucleic Acids Res 2013, 41(10):e108.
6. Butler A, Hoffman P, Smibert P, Papalexi E, Satija R. Integrating single-cell transcriptomic data across different conditions, technologies, and species. Nat Biotech 2018, 36:411-420.
7. Qiu X, Mao Q, Tang Y, Wang L, Chawla R, Pliner H A, Trapnell C. Reversed graph embedding resolves complex single-cell trajectories. Nat Methods, 2014, 14:979-982.
8. Gene Ontology Consortium et al. Gene Ontology: tool for the unification of biology. Nat Genet, 2000, 25(1):25-29.
9. Gene Ontology Consortium. The Gene Ontology Resource: 20 years and still Going strong. Nucleic Acids Res, 2019, 47(D1):330-338.
10. Mi H, Huang X, Muruganujan A, Tang H, Mills C, Kang D, Thomas P D. PANTHER version 11: expanded annotation data from Gene Ontology and Reactome pathways, and data analysis tool enhancements. Nucleic Acid Res, 45(D1):183-189.
11. Supek F, Bosnjak M, Skunca N, Smuc Tomislav. REVIGO Summarizes and Visualizes Long Lists of Gene Ontology Terms. PLOS One, 2011.

### OTHER EMBODIMENTS

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

The patent and scientific literature referred to herein establishes the knowledge that is available to those with skill in the art. All references, e.g., U.S. patents, U.S. patent application publications, PCT patent applications designating the U.S., published foreign patents and patent applications cited herein are incorporated herein by reference in their entireties. Genbank and NCBI submissions indicated by accession number cited herein are incorporated herein by reference. All other published references, documents, manuscripts and scientific literature cited herein are incorporated herein by reference. In the case of conflict, the present specification, including definitions, will control. In addition, the materials, methods, and examples are illustrative only and not intended to be limiting.

While this invention has been particularly shown and described with references to preferred embodiments thereof, it will be understood by those skilled in the art that various changes in form and details may be made therein without departing from the scope of the invention encompassed by the appended claims.

1. A population of mammalian cells, wherein at least 30% of the cells are PAX7+ myogenic progenitor cells (MPCs) derived from pluripotent stem cells in vitro.

2. The population of claim 1, which comprises at least 10,000 cells.

3. The population of claim 1, wherein the PAX7+ MPCs express one or more of CHRNA1 (Cholinergic receptor nicotinic alpha 1), NTSR1 (Neurotensin receptor 1), or FZD1 (Frizzled class receptor 1).

4. The population of claim 3, wherein the PAX7+ MPCs express at least two of CHRNA1, NTSR1, and FZD1.

5. The population of claim 3, wherein the PAX7+ MPCs express CHRNA1, NTSR1, and FZD1.

6. The population of claim 1, wherein the PAX7+ MPCs do not express one or more of FZD5 (Frizzled class receptor 5), GPR37 (G protein-coupled receptor 37), or GPR27 G (protein-coupled receptor 27).

7. The population of claim 6, wherein the PAX7+ MPCs do not express any one of FZD5, GPR37 or GPR27.

8. The population of claim 1, wherein the PAX7+ MPCs express NCAM and do not express HNK1.

9. The population of claim 1, wherein at least 35% of the cells are PAX7+ MPCs.

10. The population of claim 9, wherein at least 40% of the cells are PAX7+ MPCs.

11. The population of claim 1, wherein the pluripotent stem cells are induced pluripotent stem cells (iPSC) or embryonic stem cells (ESC).

12. The population of claim 1, wherein the cells are human cells.

13. A method for treating a degenerative muscle wasting disease or condition in a patient in need thereof, comprising injecting to the patient the population of claims of claim 1.

14. The method of claim 13, wherein the degenerative muscle wasting disease or condition is selected from the group consisting of muscular dystrophy, myopathy, a mitochondrial disease, soft tissue sarcoma, an ion channel disease, cachexia and sarcopenia.

15. The method of claim 14, wherein the degenerative muscle wasting disease or condition is Duchenne muscular dystrophy (DMD).

16. A method for producing a population of myogenic progenitor cells (MPCs), the method comprising:
differentiating a plurality of pluripotent stem cell in a medium comprising a selective inhibitor of glycogen synthase kinase 3 (GSK-3) to obtain differentiated cells;
treating the differentiated cells with an inhibitor of Notch signaling; and
expanding the differentiated cells with a fibroblast growth factor (FGF), thereby obtaining a population of MPCs expressing PAX7 (paired box protein).

17. The method of claim 16, further comprising enriching the MPCs.

18. The method of claim 17, wherein the obtained population comprises at least 30% PAX7+ MPCs.

19. The method of claim 16, wherein the selective GSK-3 inhibitor is selected from the group consisting of CHIR99021, SB216763, SB415286, BIO, and combinations thereof.

20. The method of claim 16, wherein 1) the Notch signaling inhibitor is DAPT and/or 2) the FGF is FGF-2 and FGF-8.

21. (canceled)
